(19) United States
(12) Patent Application Publication
(10) Pub. No.: US 2005/0059931 A1
(43) Pub. Date: Mar. 17, 2005

(54) Methods and apparatus for localized and semi-localized drug delivery

(75) Inventors: Michi Garrison, Half Moon Bay, CA (US); Todd J. Brinton, Menlo Park, CA (US); Peter F. Campbell, Santa Clara, CA (US); Steve Roe, San Mateo, CA (US); Stephen N. Salmon, Napa, CA (US); Paul Yock, Atherton, CA (US)

Correspondence Address: ORRICK, HERRINGTON & SUTCLIFFE, LLP, 4 PARK PLAZA, SUITE 1600, IRVINE, CA, 92614-2558, US

(73) Assignee: Venomatrix

(21) Appl. No.: 10/783,217

(22) Filed: Feb. 20, 2004

Related U.S. Application Data
(63) Continuation-in-part of application No. 10/664,171, filed on Sep. 16, 2003.

(51) Int. Cl. A61M 29/00

(52) U.S. Cl. 604/101.04

(57) ABSTRACT

A catheter system for localized or semi-localized administration of agents through the wall of a blood vessel is provided. Various catheter system constructions which use at least one expandable occluding device to create an isolated region are provided. Constructions using one catheter and one occlusion device are provided, along with constructions using two catheters and multiple occlusion devices. The catheter system may include a catheter with a variable stiffness along its length. The catheter system may also include a guide wire integrated with an inner catheter. The catheter can infuse the agent into the blood vessel in a pressure regulated manner. Methods for delivery and infusion of the agent within a blood vessel are also provided.

### CROSS-REFERENCE TO RELATED APPLICATIONS

This application is a continuation-in-part of U.S. application Ser. No. 10/664,171, filed Sep. 16, 2003, which is fully incorporated herein by reference.

### FIELD OF THE INVENTION

The present invention relates generally to intravascular drug delivery to localized and semi-localized regions. The invention includes a catheter device having one or more occluding devices, preferably balloons, associated therewith.

### BACKGROUND OF THE INVENTION

Methods for localized and semi-localized drug delivery are disclosed in Yock et al. U.S. Pat. No. 6,346,098, which is incorporated by reference, in its entirety, herein. The aforesaid Yock et al. patent describes several ways in which a pressurized system can be used to accomplish retrograde perfusion, alone or in conjunction with other modalities, e.g., energy, to cause disruption or increased porosity in a localized region of the wall of a blood vessel whereby an agent, e.g., a therapeutic substance, is caused to pass through the wall of a blood vessel to produce the desired effect in the tissue surrounding the localized delivery site. Angiogenesis and myogenesis are two particularly desirable uses of the Yock et al. method. Given the desirability of the effective use of that method, there remained a need for apparatus which would improve the effectiveness of the method and for improvements in the method itself.

It is also noted that Corday et al. U.S. Pat. Nos. 4,689,041 and 5,033,998 make use of a catheter having an occluding balloon at its distal end for retrograde venous injection of fluids into a blockaded region of the heart which has become inaccessible by reason of an occluded artery. The method of Corday et al. involves placing the balloon into the coronary sinus and directing fluid retrograde into all veins of the heart. Since the objective of Corday et al. is to deliver cardioplegic solution to the entire heart, the described system would appear to be suited for its purpose. However, it would not be useful to achieve the objectives of Yock et al. U.S. Pat. No. 6,346,098 which are centered on localized and semi-localized delivery through the wall of a blood vessel.

The patent to Glickman, U.S. Pat. No. 5,919,163, which is incorporated herein by reference, describes the use of a double balloon catheter to isolate a tumor for chemotherapy treatment.

### SUMMARY OF THE INVENTION

The apparatus of the present invention includes a catheter system for delivery of an agent, where the catheter system has one or more occluding devices, preferably balloons, which function to isolate a region within a blood vessel whereby the delivery of an agent through the blood vessel wall will take place only in the localized or semi-localized region. In one embodiment of this catheter system, at least two members, each of which may be a catheter, are used to carry an occluding device to the desired location in the blood vessel. At least one of the catheters preferably has two or more regions of variable stiffness. In this embodiment, the catheter system preferably comprises a telescoping assembly of two catheters, each provided with an occlusion device whereby the length of the isolated region may be varied. The occlusion device may be an inflatable member such as a balloon. In this event, the catheter provided with an inflatable occlusion device may be provided with an inflation lumen, which communicates with the inflatable occlusion device.

The first, or distal, inner catheter includes a distal occlusion device and is configured to move within a lumen of a second, or proximal, outer catheter having a proximal occlusion device. A desired agent can be delivered to the isolated space between the two occlusion devices from an open distal end of the outer catheter, and then infused into the localized region. In an additional embodiment, a separate lumen can be provided for infusion, preferably located within the inner catheter. In either embodiment, the sizing of the infusion lumen will depend on the infusion flow rate desired.

Many of the catheter embodiments described herein can be used to infuse an agent into regions of differing size. Depending on the particular application as well as the anatomy of the vasculature, infusion can occur to either a localized region or a larger, semi-localized region, both of which are located external to the blood vessel. For example, one location where localized delivery of the agent can occur is in a continuous blood vessel segment isolated by occlusion devices and without side branches. When the fluid pressure of the agent within the isolated space of the vessel reaches a high enough level, the vessel walls can become disrupted and allow the agent to pass through the walls and into the localized region surrounding the vessel.

Alternatively, one location where semi-localized delivery of the agent can occur is in a blood vessel segment having numerous smaller side branches or connecting vessels. These smaller vessels can limit the potential collateral escape of the agent by restricting flow of the agent to such a degree that the desired infusion pressure can be reached. Once the pressure is great enough, the smaller vessels can become disrupted, and in some case even burst, allowing the infusion agent to pass through and into the surrounding tissue or interstitium. By delivering and infusing the agent through each of these smaller vessels, a much larger, semi-localized region can be reached. This can be desirable in certain applications because it allows infusion of more of the agent over a wider area.

The catheter system of the present invention may use a coaxial or dual-lumen construction for the outer catheter and may use a tri-lumen construction for the inner catheter. In one embodiment, the system is provided with a pressure monitoring lumen in the distal catheter. This lumen extends distally along the length of the inner catheter and has a distal end, which is provided with a port, which opens into the space outside of the catheter at a location proximal to the distal occlusion device. When the two catheters are placed axially within a blood vessel, the port at the distal end of the pressure monitoring lumen is located between the two occlusion devices. The proximal end of the pressure monitoring lumen can be coupled with a pressure sensor and used to monitor pressure in the space between the two occlusion devices.

In still another embodiment, the system can be constructed such that the outer catheter with the proximal occlusion device is placed first using a guide wire and/or malleable stylet, such that this catheter acts as a guide for the inner catheter having the distal occlusion device. In certain applications, it is desirable for the outer catheter to be placed in the coronary sinus and certain physical characteristics are desirable for this purpose. These characteristics include a reinforced shaft which can transmit torque in its proximal region, which does not enter the vasculature (e.g., approximately 50 cm). The distal end is more flexible thereby enabling tracking into the venous anatomy. Additionally, the outer catheter shaft can have a pre-formed curve in its distal region, so that the catheter can be pointed in the proper direction to facilitate making a turn into the coronary sinus. A dilator can be used to substantially straighten the pre-formed curve if desired. Alternatively, the catheter shaft can be substantially straight and used in conjunction with a stylet to facilitate navigation within the coronary sinus.

The present invention also includes a system in which the inner catheter and the outer catheter are placed such that the inner catheter is placed first and acts as a rail over which the outer catheter may be advanced. In one embodiment thereof, before introducing either catheter, a guide wire is placed within the vessel and the inner catheter is advanced with the aid of the guide wire. In another embodiment thereof, the guide wire is integrated with the inner catheter and can be advanced into the vessel without the aid of an additional guiding device. The integrated guide wire can be coupled to the distal end of the inner catheter and can extend distally therefrom. The guide wire can also be curved to facilitate navigation within the vasculature. In another embodiment, the distal end of the guide wire is covered by and coupled with an atraumatic distal tip of the inner catheter.

In another embodiment of the present invention, an additional occlusion device can be provided with the inner catheter so that the inner catheter has both a distal and a proximal occlusion device enabling the inner catheter to be used without the outer catheter. This embodiment can be used to isolate a small, fixed, axial region of the blood vessel. An infusion lumen is provided within the inner catheter and connected to an aperture located between the two occlusion devices such that the desired infusion agent can be delivered to this isolated region. If the outer catheter is used, occlusion can be performed using one of the occlusion devices on the inner catheter and the occlusion device on the outer catheter.

In certain applications, the catheter system of the present invention can be used to deliver an agent to a semi-localized or localized region of the body with only one occlusion device. In one example embodiment, the inner catheter includes an axially indented occlusion device, which is preferably a balloon, that can create an isolated region of the blood vessel corresponding to the shape of the indentation. When the balloon is inflated, it contacts the entire circumference of the blood vessel along an axial length, except for the region of the blood vessel adjacent where the indented portion is located. The isolated region corresponds to where the indented portion of the balloon does not contact the vessel wall. The axial indentation is preferably located in the middle section of the balloon. A portion of the indentation preferably contacts the inner catheter such that an aperture can be provided in the inner catheter to deliver the infusion agent to the isolated region and into the localized region of the body.

This embodiment can also be used to deliver the agent to a semi-localized region of the body. For instance, the axial indentation of the balloon can be aligned within the blood vessel such that the region of the blood vessel adjacent to the indented portion of the balloon includes a communicative junction with a second blood vessel, i.e., an opening for blood to flow into or out of a second blood vessel. This second blood vessel preferably branches into a plurality of smaller vessels that form a flow restricting configuration that limits any potential collateral escape and allows the agent to be delivered at a pressure sufficient to infuse the agent through the numerous smaller vessels and into the larger, semi-localized region.

In other embodiments, a substantially isolated region is created using only one occlusion device located on the outer catheter. These embodiments are preferable in applications where the vessel where infusion is to occur has a flow restricting configuration that limits any potential collateral escape of the agent. The occlusion device on the outer catheter is expandable to occlude the vessel and create a substantially isolated blood vessel region defined by the downstream, or proximally located occlusion device and the upstream, or distally located flow restricting configuration of the vessel. The agent can then be delivered to the isolated region through an infusion means located distal to the occlusion device.

In an example embodiment of a catheter system having one occlusion device, the outer catheter can include an inner tubing, middle reinforced tubing and an outer tubing. The outer tubing can include a first occlusion balloon that is expandable to create a substantially isolated blood vessel region. The middle tubing is preferably coupled with the outer tubing and extends within the outer tubing. The space between the middle tubing and the outer tubing defines a first lumen configured to pass an inflation medium to the first occlusion balloon. The inner tubing is preferably coupled with the middle tubing and extends within the middle tubing. The space between the middle tubing and the inner tubing defines a second lumen configured to deliver an infusion agent to an open distal end of the middle tubing. The space within the inner tubing defines a third lumen configured to monitor pressure within the isolated blood vessel region.

The catheter system of the present invention can also include a pressure regulator for regulating the pressure of an infusion agent in the isolated blood vessel region. The pressure regulator can be incorporated in an injection device, or it can be coupled between an injection device and the catheter system. The pressure regulator can be an accumulator type pressure regulator or can regulate the allowable fluid flow rate directly, such as with a valve and the like. The pressure regulator can also use fluid pressure feedback from the infusion site to regulate the fluid flow rate at the injection device.

Infusion pressure can be regulated in at least two ways. Infusion pressure can be regulated passively, e.g., by including a biased reservoir that controls flow through a regulator at the input or output of the catheter system based on the fluid pressure within the reservoir. Infusion pressure can also be regulated actively, e.g., by monitoring the infusion pressure at the infusion site with a fluid pressure feedback and using this feedback pressure to control flow through the regulator located at the input to the catheter system. At least partial passive pressure regulation is preferable in order to prevent the fluid pressure at the infusion site from exceeding a maximum desired pressure that might injure the patient.

The balloons are preferably fabricated from a compliant material and have a variable diameter depending on inflation volume and/or pressure. Such materials include elastic polymers such as elastomeric polyurethane, silicone polymers, synthetic rubbers such as polyneoprene, neoprene and polybutylene, thermoplastic elastomers and other elastic materials well known to those skilled in the art. The balloons can be configured with any desired shape, such as spherical or cylindrical.

Radio opaque markers may be added to one or both catheters to mark desired points on catheters, e.g., the distal region of each catheter and/or the proximal position of the distal occlusion device. Also, radio opaque dye can be injected through the annular space during placement of the catheter. The use of radio opaque markers or dye will help catheter positioning and accurate measurement of the infusion space. Furthermore, radio opaque dye can be introduced with the agent, or prior to delivery of the agent, to monitor the infusion of the agent into the localized or semi-localized region.

The present invention also provides numerous methods for infusing an agent to a localized or semi-localized region of the body. These methods are capable of use with each of the various embodiments of the catheter system described above.

### DETAILED DESCRIPTION OF THE DRAWINGS

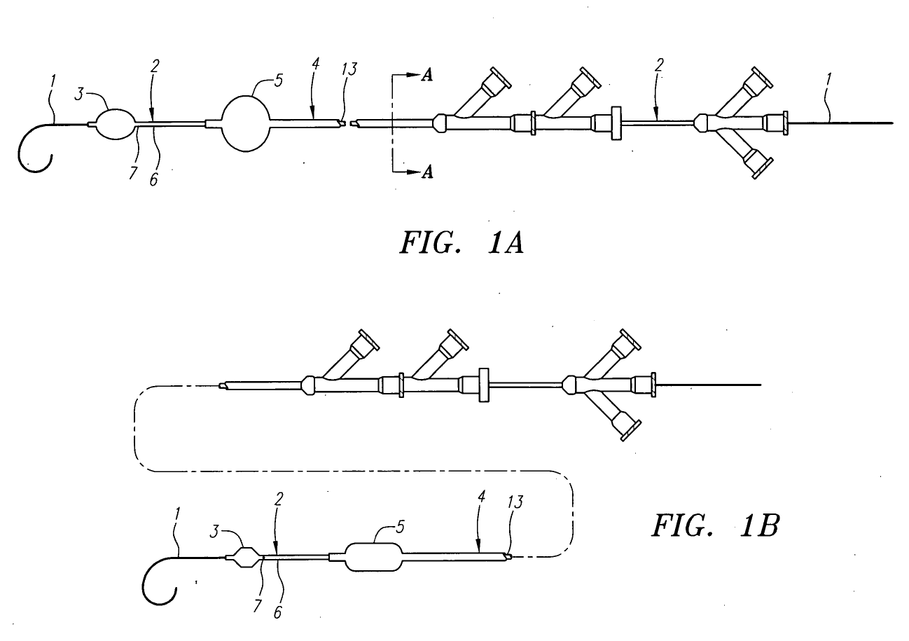

FIGS. 1A-B illustrate schematic views of example embodiments of the catheter system of the present invention.

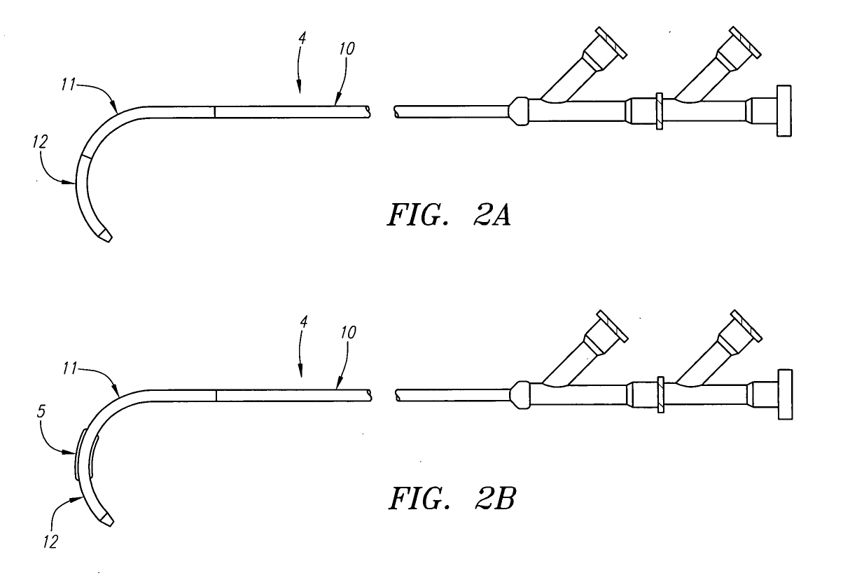

FIGS. 2A-B illustrate the regions of one example embodiment of the outer catheter, which have different stiffnesses.

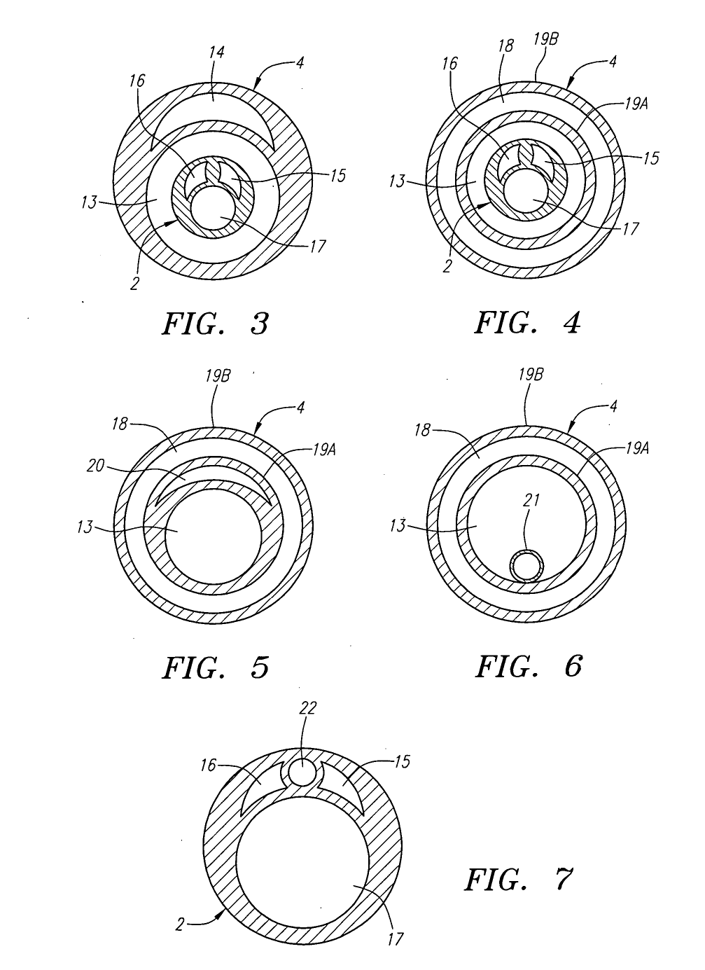

FIG. 3 illustrates the transverse cross sections of an example dual-lumen embodiment of the outer catheter and a tri-lumen embodiment of the inner catheter of the present invention.

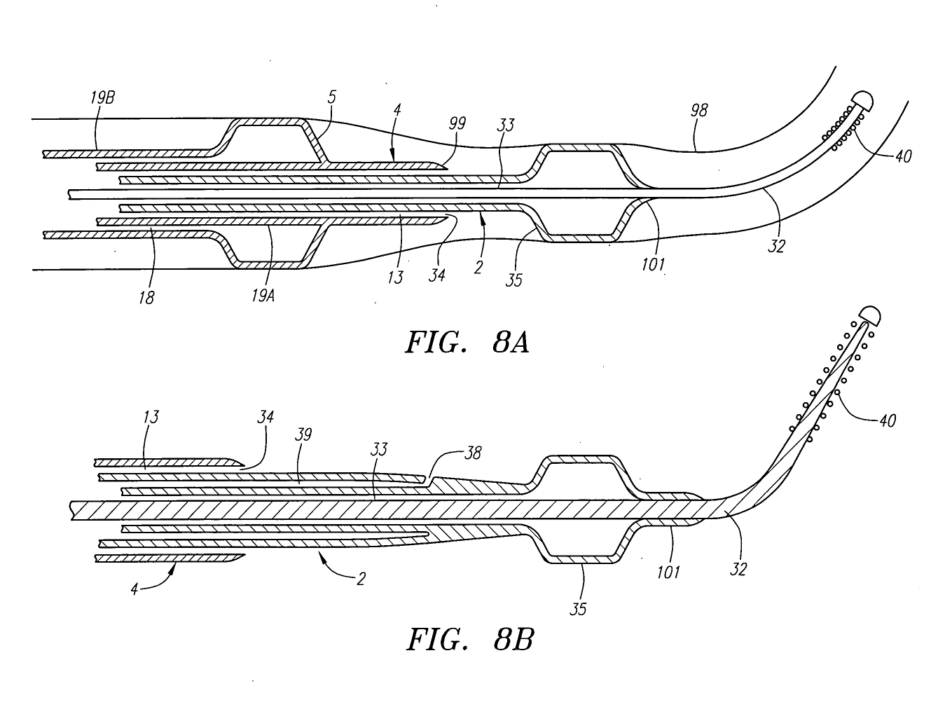

FIGS. 4, 5, 6 and 7 illustrate the transverse cross sections of catheters which may be used according to the present invention and which have different lumen configurations.

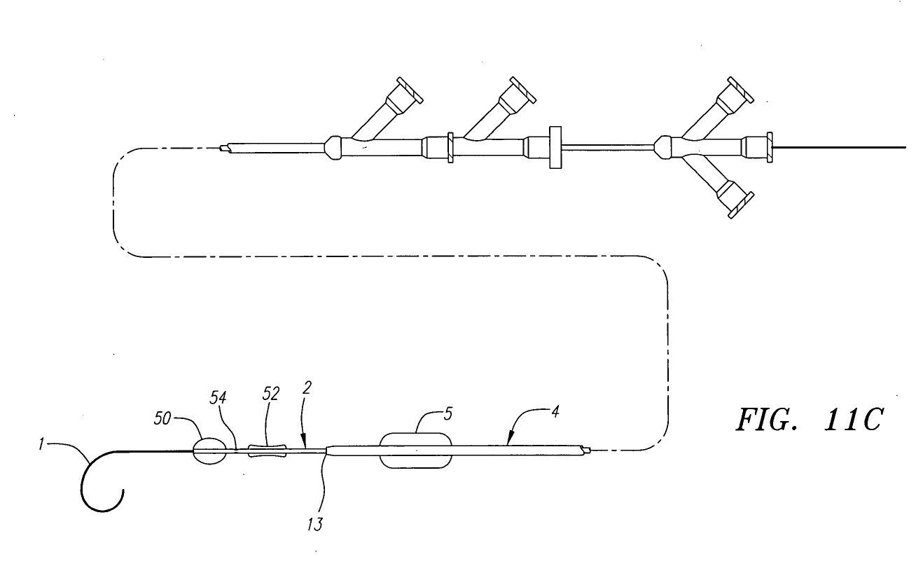

FIGS. 8A-B illustrate an alternate example embodiment in which an integrated guide wire and inner catheter are provided with an expandable occlusion device.

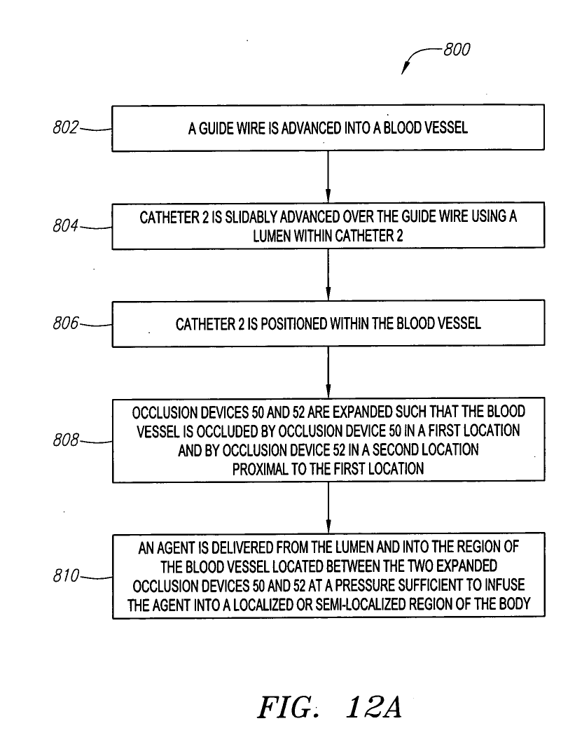

FIG. 9 illustrates another alternate example embodiment in which a guide wire is integrated with the inner catheter.

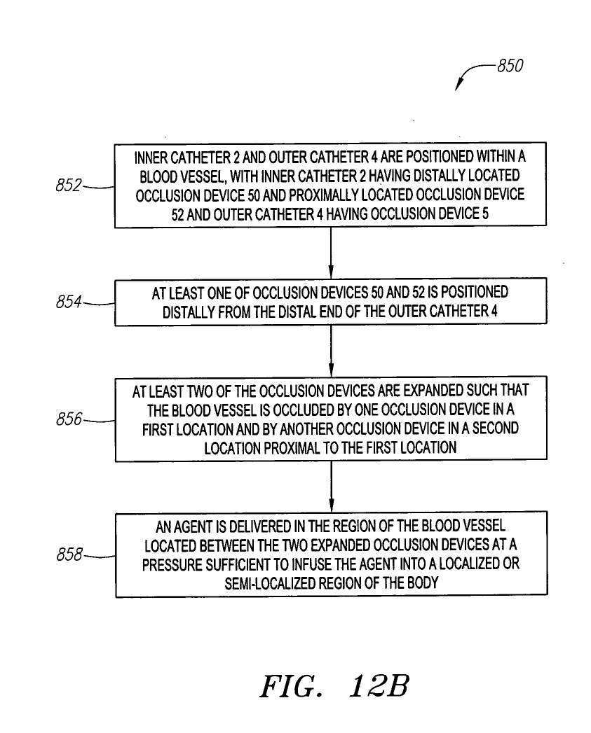

FIG. 10 illustrates an example method of delivering an infusion agent using the catheter system of the present invention.

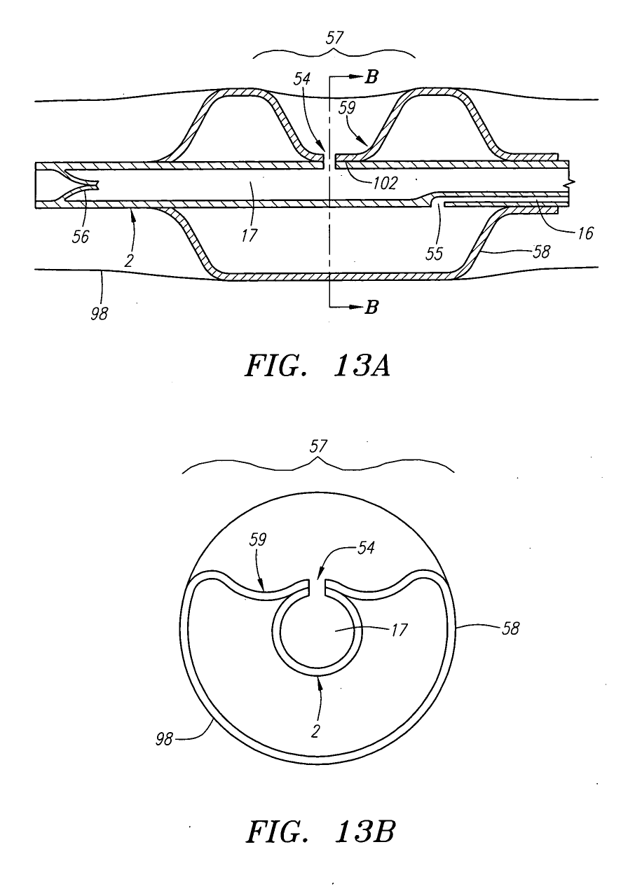

FIGS. 11A-C illustrate the catheter system with an example embodiment of the inner catheter.

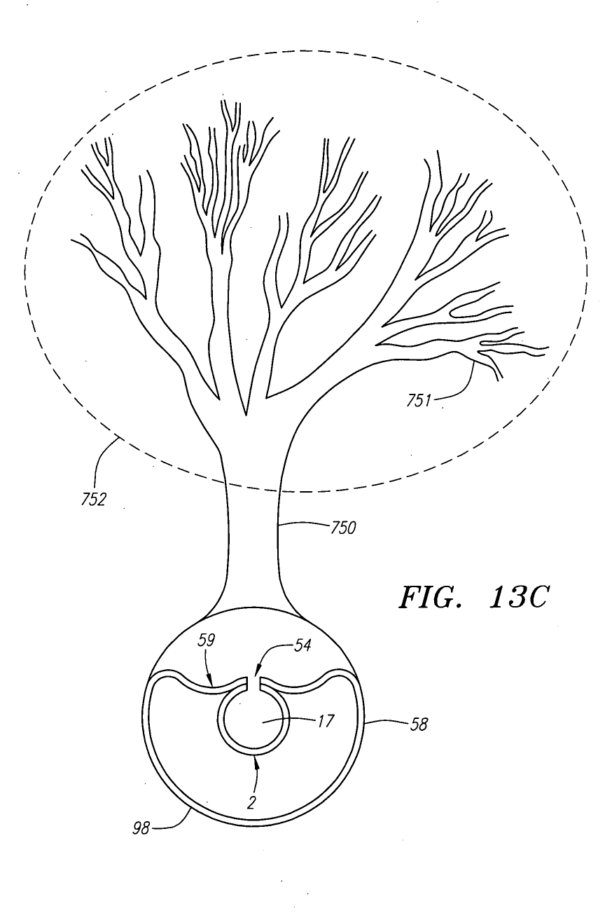

FIGS. 12A-B illustrate additional example methods of delivering an infusion agent using the catheter system of the present invention.

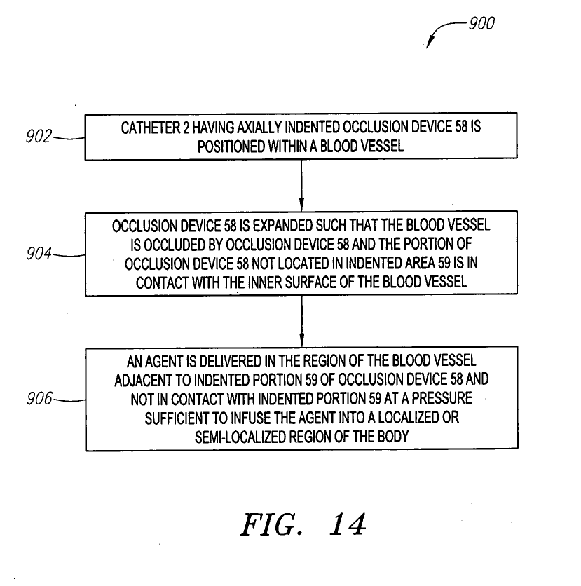

FIGS. 13A-C illustrate cross-sectional views of the catheter system with another example embodiment of the inner catheter.

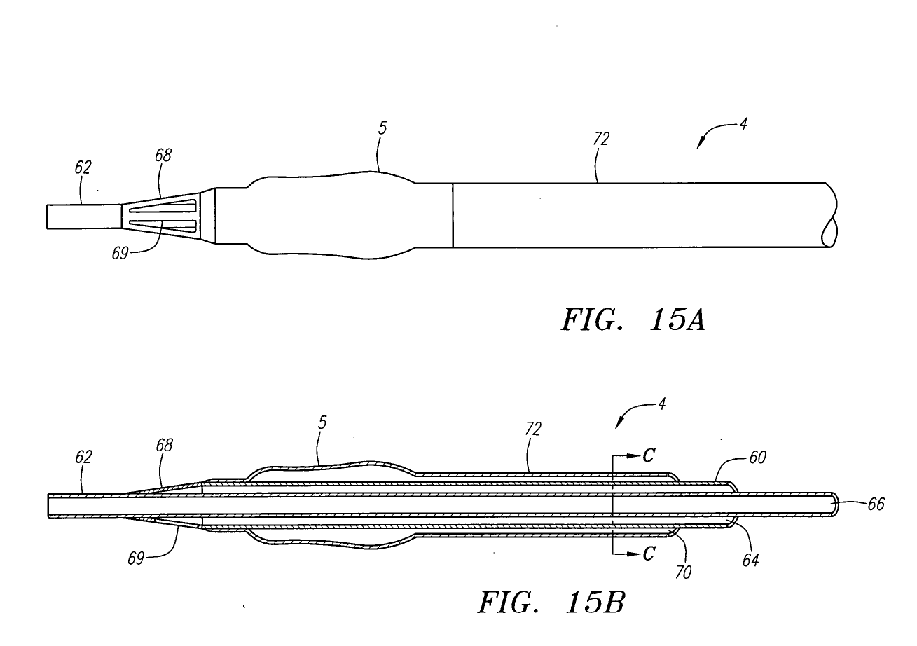

FIG. 14 illustrates another example method of delivering an infusion agent using the catheter system of the present invention.

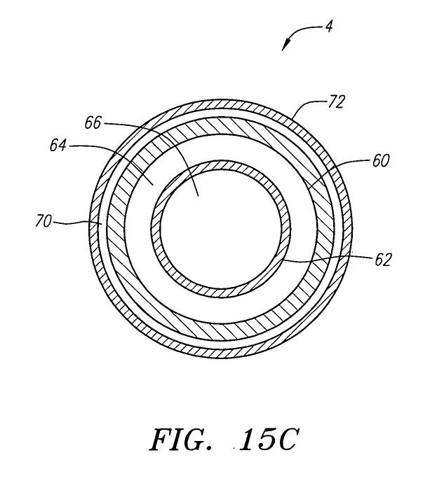

FIGS. 15A-C illustrate the catheter system with additional example embodiments of the outer catheter.

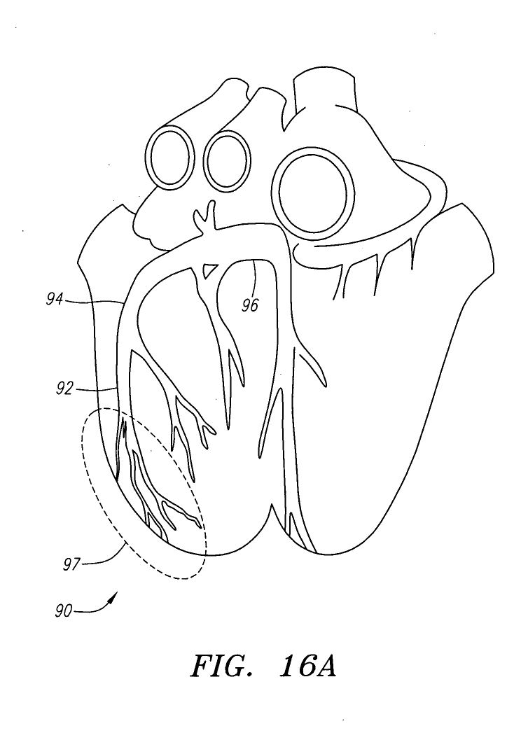

FIG. 16A illustrates an example human heart and coronary venous system for applications using the catheter system.

FIG. 16B illustrates another example method of delivering an infusion agent using the catheter system of the present invention.

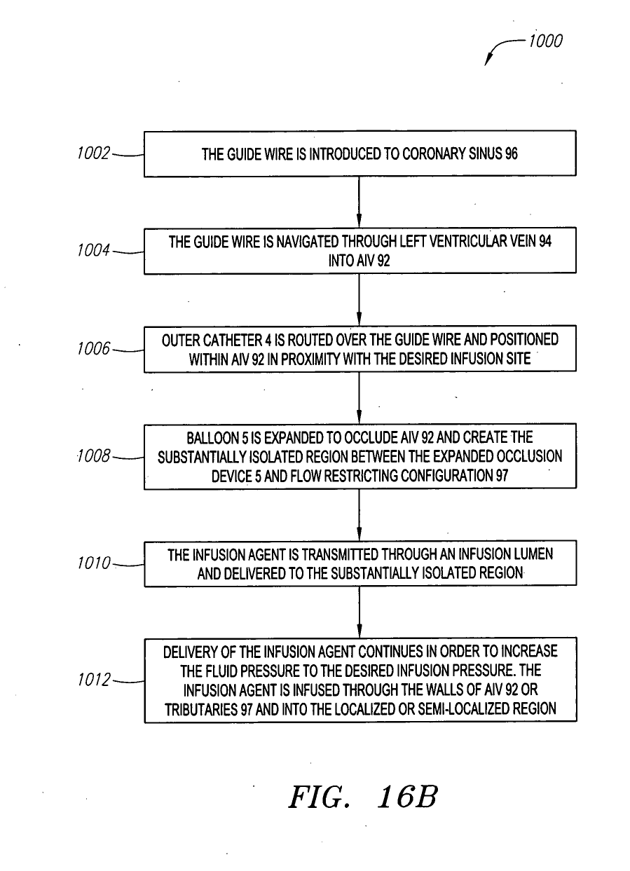

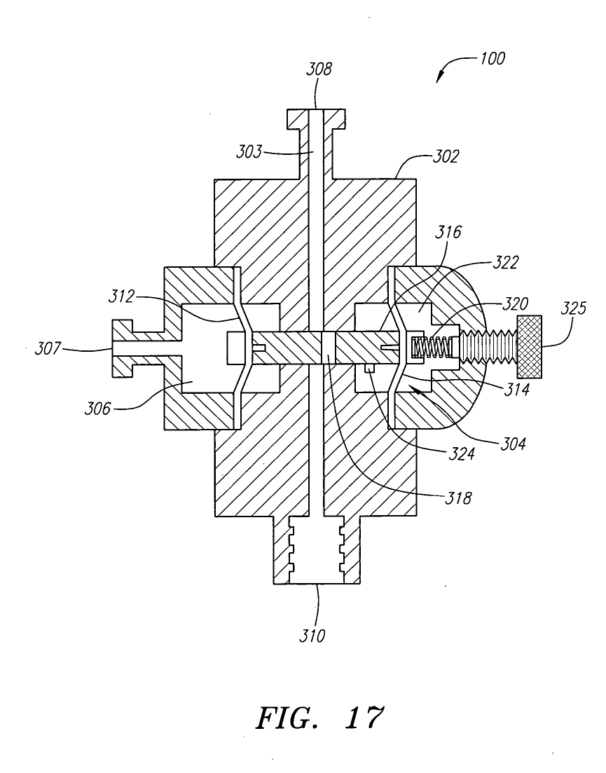

FIGS. 17, 18A-C, 19A-C, 20A-C, 21, 22, 23A-B, 24A-D and 25A-B illustrate example embodiments of a pressure regulator which may be used in conjunction with the present invention.

### DETAILED DESCRIPTION OF THE INVENTION

As can be seen from FIGS. 1A-B, one example embodiment of the present invention comprises two catheters, each of which is provided with an occlusion device. The catheter system is constructed such that it can pass over guide wire 1. Inner catheter 2 carries distal occlusion device 3. Similarly, outer catheter 4 carries proximal occlusion device 5. One of skill in the art will readily recognize that any occlusion device can be used with the present invention and, accordingly, the present invention is not limited to any particular type or style of occlusion device. Here, occlusion devices 3 and 5 are balloons. Occlusion balloons can be shaped according to the needs of the application. In FIG. 1A, occlusion balloons 3 and 5 are spherical in shape, while in FIG. 1B, occlusion balloons 3 and 5 are cylindrical in shape. The cylindrical shape depicted in FIG. 1B is preferred in order to increase the surface area in contact between the balloons 3 and 5 and the vessel. The increased contact allows balloons 3 and 5 to more adequately occlude the vessel. The section 6 of inner catheter 2 can be provided with infusion means, e.g., ports, through which a desired agent, e.g., cells, may be delivered to and administered to the patient through the blood vessel wall surrounding region 6 in a manner such as the one disclosed in Yock U.S. Pat. No. 6,346,098. As further shown in FIGS. 1A-B, the distal region of inner catheter 2 may also be provided with a pressure monitoring port 7, which preferably holds a static column of fluid for use in measuring the pressure of the infusion agent. In another embodiment, the infusion agent can be delivered through main lumen 13, which is within outer catheter 4 and houses inner catheter 2. Infusion through main lumen 13 eliminates the need for a separate infusion means in section 6, which can optionally be eliminated. In one example embodiment, the annular free space in main lumen 13 between inner catheter 2 and outer catheter 4 is approximately 0.015 inches.

FIG. 2A is a simplified illustration of outer catheter 4 of FIG. 1. Details of the catheter, such as the balloon, have been omitted for purposes of clarity. The catheter system preferably has two or more regions of varying stiffness along its length. For instance, in FIG. 2A, that outer catheter 4 has a relatively stiff proximal region indicated by numeral 10, a softer intermediate region indicated by numeral 11 and a still softer distal region indicated by the numeral 12. The purpose of these three regions of different stiffness is to provide pushability and torque ability with the relatively stiff proximal region 10 and track-ability with the softer intermediate and distal regions 11 and 12. Each region can have a fixed stiffness along its length or it can have a varying stiffness. For instance, in FIG. 2A, each region 10-12 is shown having a definite boundary, however the regions need not have definite boundaries and can have transitions of variable stiffness. Outer catheter 4 can also have a pre-formed curve for facilitating navigation within the vasculature. A dilator can be used to substantially straighten the pre-formed curve as desired. Alternatively, a stylet can be used to provide instead of a pre-formed curved region or an addition to a pre-formed curved region as desired. The stylet is preferably composed of a shape memory alloy such as nitonol.

This construction facilitates deployment of the distal region of the catheter through the coronary sinus into distal venous branches of the patient, which is desirable when treatment will be for purposes of angiogenesis or myogenesis. In a preferred embodiment of the present invention, cells which will promote angiogenesis or myogenesis are delivered to a localized region of the heart.

As shown in FIGS. 4-6, in one example embodiment of a coaxial catheter, outer catheter 4 is fabricated with inner tubing 19A and an outer tubing 19B. Inner tubing 19A can include an inner layer and outer layer, or jacket. Inner tubing 19A can further include a structure for reinforcement (not shown), such as a metal braid located between the inner and outer layers. The reinforcement structure can extend along any desired length of outer catheter 4, preferably along the entire length to provide adequate torque and push/pull response. The three regions 10-12 of outer catheter 4 can be fabricated in any manner that allows the relative stiffness of each region to vary. In a preferred embodiment, the outer layer in each region 10-12 of outer catheter 4 is composed of a material with a different durometer measurement of hardness, where the material used in intermediate region 11 is relatively harder than that of distal region 12, and the material used in proximal region 10 is relatively harder than that of intermediate region 11. Other manners of varying the stiffness of outer catheter 4 are also contemplated herein, such as by varying the length of the reinforcement structure, or by varying the degree of reinforcement provided by the reinforcement structure along the length of outer catheter 4.

FIG. 2B illustrates an example embodiment of outer catheter 4 with occlusion balloon 5 attached thereto. Although balloon 5 can be placed on any region of outer catheter 4, balloon 5 is preferably placed on the intermediate region 11 in order to allow balloon 5 to inflate without collapsing outer catheter 4. When balloon 5 is inflated, it applies pressure to the circumference of outer catheter 4 and can cause the outer catheter 4 to collapse or “pinch off.” The susceptibility of outer catheter 4 to collapse depends mainly on the stiffness, or rigidity, of outer catheter 4 and the inflation pressure of balloon 5, where a lower stiffness, or rigidity and/or a higher inflation pressure tend to increase the likelihood of collapse. Placing balloon 5 on the intermediate region 11 allows greater inflation pressures than if balloon 5 were placed on the softer region 12. Accordingly, placement of balloon 5 on stiffer region 10 would allow even greater inflation pressures. Also, by decreasing the compliance of balloon 5, the required inflation pressure can be reduced and any collapse of outer catheter 4 can be more easily avoided. However, this may not be a viable option for applications that require a low inflation pressure to create adequate occlusion.

In a preferred embodiment of the present invention, the inner catheter 2 is slidably associated with outer catheter 4 such that the space between balloon 3 and balloon 5 can be varied according to the circumstances of the desired treatment. Published U.S. patent application 2002/0188253, which is incorporated herein by reference, discloses a dual balloon system in which the catheters are slidable with relation to each other to thereby vary the space between the balloons as desired.

One of skill in the art will readily recognize that the placement of balloon 5 and the lengths of each region 10, 11 and 12 can be varied based on the needs of the individual application. For instance, an application may be very susceptible to pinch off in which case balloon 5 can be placed on the relatively stiff proximal region 10. Also, an application may require relatively smaller distance between balloons 3 and 5. In this case, balloon 5 can be placed on softer region 12 so long as the inflation pressure and the region durometer hardness is not such as to cause outer catheter 4 to collapse. Also, balloon 5 can be placed on the relatively stiff proximal region 10 and the relative lengths of each region 10 and 11 can be shortened, so long as the catheter retains sufficient track-ability to allow advancement into the target region of the patient.

FIGS. 3-7 are cross-sectional illustrations taken along A-A of FIG. 1A. FIGS. 3 and 4 illustrate different example embodiments of outer catheter 4 as well as other details of the present invention. In FIG. 3, the shaft of the outer catheter 4 is shown in a dual-lumen configuration with main lumen 13 and inflation lumen 14. In this embodiment, the inflation medium for balloon 5 is passed through lumen 14. In this Figure, inner catheter 2 is also shown and has lumen 17 through which a guide wire or stylet may pass, as well as pressure monitoring lumen 15 and balloon inflation lumen 16. The proximal end of pressure monitoring lumen 15 can be coupled with a pressure sensor to measure the pressure at the infusion site. In addition, as will be described below, lumen 15 can be coupled with a pressure regulator and used to provide a fluid pressure feedback.

In FIG. 4, catheter 4 is provided in a coaxial configuration such that it has main lumen 13 and annular space 18 which constitutes a passageway for the balloon inflation medium. Annular space 18 is formed by the outer surface of inner tubing 19A which is spaced from the inner surface of outer tubing 19B of catheter 4. Inner tubing 19A and outer tubing 19B are preferably connected and sealed at the distal end of outer catheter 4 allowing annular space 18 to extend distally along a desired length of outer catheter 4 and terminate distal to an inflation aperture for balloon 5. In this embodiment, the structure of inner catheter 2 remains the same as that illustrated in FIG. 3.

In the embodiments of FIGS. 3 and 4, the main lumen 13 will be used for tracking over a guide wire, stylet, dilator, or previously-installed catheter, for guiding the insertion or withdrawal of inner catheter 2, and as a conduit for the infusion medium used to deploy the agent through the blood vessel wall at the desired location.

The infusion pressure in the isolated blood vessel region is preferably measured with the pressure monitoring lumen 15. However, the infusion pressure can also be calculated from the pressure in main lumen 13 when the agent is being delivered, based on the flow rate, viscosity of solution, flow resistance of the catheters 2 and 4 and assuming steady state flow. If the pressure is measured in this manner, the pressure monitoring lumen 15, illustrated in FIGS. 3 and 4, can be eliminated altogether. Use of the pressure monitoring lumen 15 is preferred for increased accuracy and reliability in pressure monitoring.

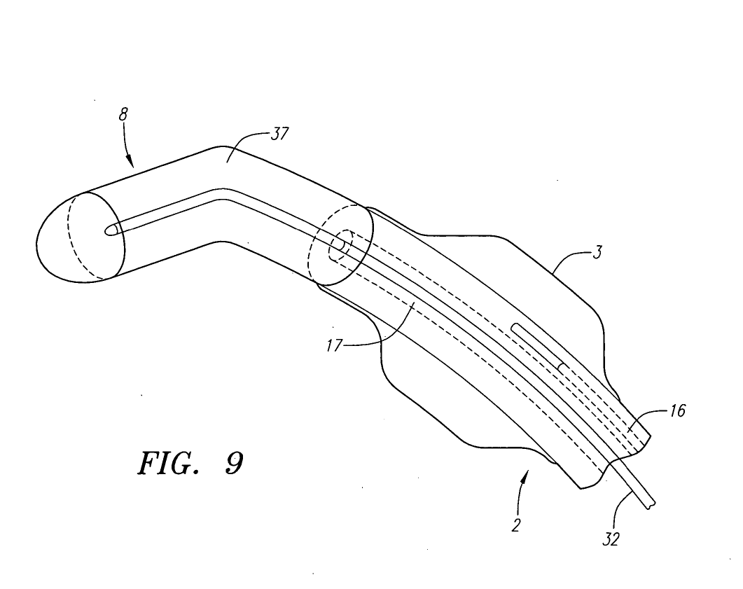

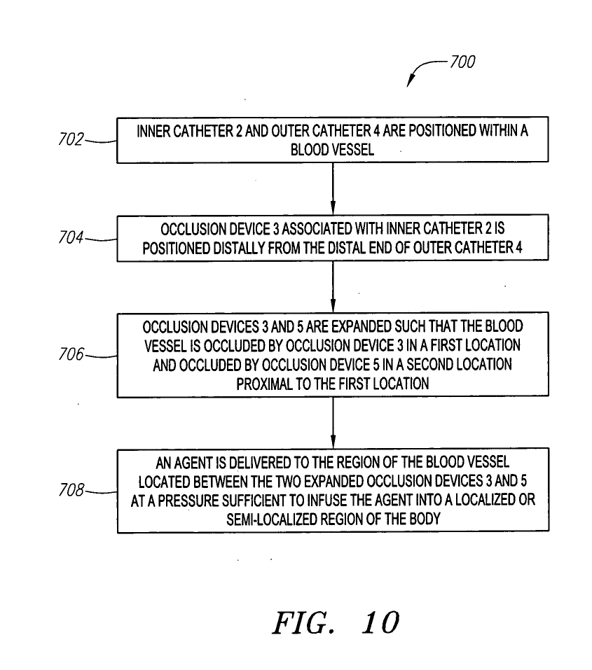

FIGS. 5 and 6 show alternate embodiments of the coaxial configuration of outer catheter 4. As shown in FIG. 5, catheter 4 is provided with an additional lumen 20 which may be used for infusion or such other purposes as may be desired. In FIG. 6, outer catheter 4 is provided with a small tube 21 which may be fabricated from any suitable metal or polymer material, e.g., stainless steel, nickel-titanium alloys, polyimides, and may serve as an additional infusion device or for such other purposes as may be desired. Tube 21 is preferably sized to allow inner catheter 2 to be slidably received within outer catheter 4.

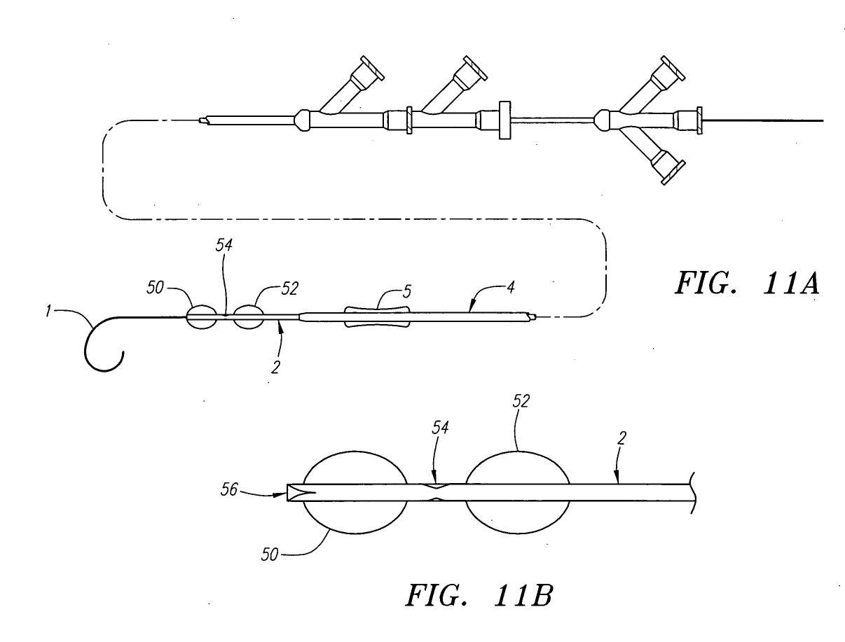

FIG. 7 illustrates, in cross section, a further embodiment of inner catheter 2 which is provided with an additional lumen 22 which may be used for infusion or such other purposes as may be desired. Other purposes include, but are not limited to, the introduction of an intravascular device, the delivery of other agents or the application of suction and the like.

All of the catheters shown herein may be circular in cross section or may have other shapes such as elliptical or irregular.

Two conventional methods could be employed to advance a catheter to the target vessel. In the first method, a guide wire is first advanced to the target vessel and then the catheter is advanced over the guide wire. In order to do this, the catheter must have an open distal end. However, if there is not a smooth tapered transition between the guide wire and the open end, then the open end can skive, or scratch, the interior of the blood vessels as the catheter is advanced. In the second method, a guide wire is first advanced to the target vessel and then a guiding catheter is advanced over the guide wire into proximity with the target vessel. The guide wire is then removed and the catheter is advanced within the guiding catheter to the target vessel. Again, there is a risk of skiving because the guiding catheter must have an open distal end to be advanced along the guide wire. FIGS. 8A-B and 9 illustrate example embodiments of the catheter system of the present invention that improve over these conventional systems and methods.

FIG. 8A illustrates an example embodiment of the present invention shown within blood vessel 98. Here, guide wire 32 is integrated with, i.e., attached to, inner catheter 2. Integrated guide wire 32 allows inner catheter 2 to be more easily advanced to the target blood vessel segment. Guide wire 32 is coupled with inner catheter 2, e.g., in the region indicated by reference numeral 101, in the distal region of inner catheter 2 to allow torque transfer between the two and also facilitate the pushability and pullability of the integrated device. In the example embodiment of FIG. 8A, the annular space between guide wire 32 and inner catheter 2, where they are not attached, forms lumen 33. Occlusion device 35, which in this embodiment is a balloon, is coupled with guide wire 32 and inner catheter 2 such that the inflation medium for balloon 35 can be transmitted through lumen 33. Preferably, inner catheter 2 is composed of a polymer, and balloon 35 is integrally coupled with inner catheter 2 in a manner sufficient for the needs of the application, such as heat welding and the like. Occlusion device 35 and inner catheter 2 are preferably coupled such that they form a continuous outer jacket over guide wire 32, with the annular space between the jacket and guide wire 32 forming lumen 33.

Also shown is outer catheter 4, which has a coaxial configuration and is deployed over inner catheter 2, also in a coaxial configuration. Outer catheter 4 includes inner tubing 19A and outer tubing 19B configured in a coaxial manner. Outer catheter 4 also includes occlusion device 5, which is depicted as a balloon in this embodiment. Lumen 13 is located within inner tubing 19A and is preferably used to deliver the desired infusion agent through open distal end 34. Annular lumen 18 is located between inner tubing 19A and outer tubing 19B and is preferably used to transmit the inflation medium to balloon 5. Balloon 5 can also be integrally coupled with outer catheter 4 in a manner similar to that described above.

Atraumatic tip 40 is located at the distal end of guide wire 32. Atraumatic tip 40 is softer than guide wire 32 and facilitates introduction of guide wire 32 into the blood vessel in an atraumatic fashion. In the example embodiment depicted here, atraumatic tip 40 is a springform tip. Springform tip 40 is preferably a coiled wire placed over a tapered end of guide wire 32 and can be optionally used as a radio opaque marker. The distal end 99 of outer catheter 4 is preferably beveled to reduce the risk of skiving. Here, infusion of the agent occurs in the localized region surrounding the isolated segment of vessel 98. However, it should be noted that the presence of one or more additional, side-branching vessels forming a flow restricting configuration in the isolated region of vessel 98 can allow infusion to occur in a larger semi-localized region.

FIG. 8B illustrates another example embodiment of the present invention similar to FIG. 8A. In this embodiment, as in FIG. 8A, inner catheter 2 is in a coaxial configuration. Inner catheter 2 includes coaxial pressure monitoring lumen 39 and atraumatic tip 40. Coaxial pressure monitoring lumen 39 preferably extends along the length of guide wire 32 and has a distal aperture 38, which is coupled by lumen 39 to a pressure regulator 100, such as those depicted in FIGS. 14-18B, or other pressure monitoring device at the proximal end of lumen 39. Pressure monitoring lumen 39 is in fluid communication with the infusion site through pressure monitoring aperture 38. Also shown is the distal end of outer catheter 4, which is deployed over inner catheter 2. Outer catheter 4 includes lumen 13, which can be used to deliver the desired infusion agent, as discussed with regard to FIG. 8A. Although not shown in FIG. 8B, outer catheter 4 also includes an occlusion device 5 for occluding the vessel in a location proximal to occlusion device 35.

In FIGS. 8A-B, the coupling of inner catheter 2 to guide wire 32 eliminates the clinical step of inserting the inner catheter 2 over a previously inserted guide wire, which simplifies the overall medical procedure. Here, both the inner catheter 2 can be inserted directly and navigated to the desired location with the aid of integrated guide wire 32. In addition, there is no open distal end of inner catheter 2 that can skive the interior of the blood vessels.

FIG. 9 illustrates another example embodiment of the catheter system. Here, inner catheter 2 is in a dual-lumen configuration having guide wire lumen 17 and inflation lumen 16. Inner catheter 2 is integrated with guide wire 32 and includes distal tip 8, which covers the distal end of guide wire 32. Distal tip 8 preferably eliminates or reduces any stepped transition between the exterior of guide wire 32 and the distal end of inner catheter 2 and reduces the risk of skiving. Distal tip 8 is located at the most distal region of inner catheter 2. Distal tip 8 can be the most distal portion of inner catheter 2 and composed of the same material as inner catheter 2. Alternatively, distal tip 8 can be a separate catheter section bonded to, or coupled with an open end of inner catheter 2. In this case, distal tip 8 can optionally be composed of a separate material. For instance, if visibility of distal tip 8 to an imaging device is desired, then distal tip 8 can be fabricated accordingly, such as by making distal tip 8 from a radio opaque material and the like. Furthermore, distal tip 8 can be made of an atraumatic material that facilitates insertion of guide wire 32 into a blood vessel.

In FIG. 9, distal tip 8 is made of a imaging-visible material thermally bonded to the distal end of inner catheter 2. Guide wire 32 is preferably disposed within lumen 17 of inner catheter 2. In this embodiment, lumen 17 extends distally along the length of inner catheter 2 up until distal tip 8, where lumen 17 ends. In a preferred embodiment, guide wire 32 is coupled with, i.e., attached to, inner catheter 2 along substantially the entire length of distal tip 8, to allow for sufficient coupling strength between inner catheter 2 and guide wire 32.

FIG. 9 also illustrates distal occlusion balloon 3 and balloon inflation lumen 16. Additional lumens can be included within inner catheter 2 as desired. Preferably, balloon 3 is located proximal to distal tip 8. This can allow distal tip 8 to be configured to facilitate navigation through blood vessels. Here, distal tip 8 is shown with bend 37 to facilitate the advancement of inner catheter 2 through curved blood vessels. Distal tip 8 can be configured in any shape or geometry, including having multiple bends or curves, to further facilitate navigation.

FIG. 10 depicts infusion method 700, which is one preferred method of infusing an agent into a localized or semi-localized region of the body. Method 700 can be used with any of the two-catheter embodiments described herein. In this preferred embodiment, at 702, inner catheter 2 and outer catheter 4 are positioned within a blood vessel. This can occur with the aid of a previously positioned guide wire, or it can occur with the aid of guide wire 32 integrated with inner catheter 2. Furthermore, if a guide wire is positioned first, either inner catheter 2 or outer catheter 4 can be first advanced over the guide wire. If inner catheter 2 is advanced first, then inner catheter 2 acts as a rail over which outer catheter 4 can be advanced. Likewise, if outer catheter 4 is advanced first, then inner catheter 2 can be advanced within outer catheter 4. If a guide wire is used, preferably, a large guide wire, such as a 0.035″ guide wire and the like, is used for positioning of outer catheter 4 first and preferably a small guide wire, such as a 0.014″ or 0.018″ guide wire and the like, is used for positioning of inner catheter 2 first.

Next, at 704, occlusion device 3 associated with inner catheter 2 is positioned distally from the distal end of outer catheter 4. Then, at 706, occlusion devices 3 and 5 are expanded such that the blood vessel is occluded by occlusion device 3 in a first location and occluded by occlusion device 5 in a second location proximal to the first location. The two occlusion devices 3 and 5 can be expanded in any order or simultaneously as desired. Finally, at 708, an agent is delivered to the region of the blood vessel located between the two expanded occlusion devices 3 and 5 at a pressure sufficient to infuse the agent into a localized or semi-localized region of the body.

Integration of inner catheter 2 with guide wire 32 creates numerous advantages and gives the catheter system of the present invention added flexibility in implementation. However, inner catheter 2 can be provided in multiple other configurations, each providing additional advantages and added flexibility in the implementation of the catheter system. For instance, the catheter system can be configured to occlude small fixed lengths of a target blood vessel, e.g., by adding a second occlusion device to inner catheter 2. FIGS. 11A-C depict example embodiments of the catheter system where inner catheter 2 is provided with two occlusion devices, distal occlusion device 50 and proximal occlusion device 52. This configuration is preferably used to occlude and isolate a relatively short, fixed length of a blood vessel region between the two devices 50 and 52. In this embodiment, occlusion devices 50 and 52 are balloons and are spaced closely together for applications requiring infusion in a very limited fixed space, for instance on the order of 5 millimeters (mm). Occlusion balloon 5 on outer catheter 4 is left uninflated during occlusion by devices 50 and 52.

The infusion agent is preferably transmitted through guide wire lumen 17 (not shown) of inner catheter 2, and delivered from an aperture, or skive 54 that is located between balloons 50 and 52. Guide wire 1 is preferably removed from within guide wire lumen 17 before infusion takes place. FIG. 11B is a cross-sectional view of inner catheter 2 depicting one-way valve 56, which is used to seal the distal end of guide wire lumen 17 once guide wire 1 is removed. Preferably, valve 56 is a small duck-bill style valve that can be sealed by the fluid pressure within lumen 17. Valve 56 preferably contains two proximally extending flaps that are pressed together by the force of the agent during delivery and infusion. In an alternative embodiment, guide wire 32 is integrated with inner catheter and sealed at the distal end, in a manner similar to that depicted in FIGS. 8A-B.

Because occlusion is accomplished using only the two occlusion devices 50 and 52 located on inner catheter 2, applications using this embodiment can optionally eliminate outer catheter 4 and perform occlusion with only inner catheter 2. However, in some applications, the added ability to also occlude a variable length of the vessel may be desired, in which case both outer catheter 4 and inner catheter 2 are used. FIG. 11C depicts an embodiment where both catheters 2 and 4 are used. Here, to occlude a variable length of the blood vessel, only occlusion balloon 50 on inner catheter 2 is inflated along with occlusion balloon 5 on outer catheter 4. Proximal occlusion balloon 52 is left uninflated. The desired length of the blood vessel can be isolated by varying the distance between distal occlusion balloon 50 and balloon 5. In this embodiment, the infusion agent can be optionally delivered via aperture 54 or main lumen 13.

FIG. 12A depicts infusion method 800, which is one preferred method of infusing an agent into a localized or semi-localized region of the body. Method 800 can be used with any of the catheter embodiments described herein having two or more occlusion devices. In this preferred embodiment, at 802, a guide wire is advanced into a blood vessel. Then, at 804, catheter 2 is slidably advanced over the guide wire using a lumen within catheter 2. Next, at 806, catheter 2 is positioned within the blood vessel. At 804, occlusion devices 50 and 52 are expanded such that the blood vessel is occluded by occlusion device 50 in a first location and by occlusion device 52 in a second location proximal to the first location. Finally, at 806, an agent is delivered from the lumen and into the region of the blood vessel located between the two expanded occlusion devices 50 and 52 at a pressure sufficient to infuse the agent into a localized or semi-localized region of the body.

FIG. 12B depicts method 850, which is one preferred method of infusing an agent into a localized or semi-localized region of the body. Method 850 can be used with any of the catheter embodiments described herein having two or more occlusion devices. In this preferred embodiment, at 852, inner catheter 2 and outer catheter 4 are positioned within a blood vessel, with inner catheter 2 having distally located occlusion device 50 and proximally located occlusion device 52 and outer catheter 4 having occlusion device 5. Then, at 854, at least one of occlusion devices 50 and 52 is positioned distally from the distal end of the outer catheter 4. Next, at 856, at least two of the occlusion devices are expanded such that the blood vessel is occluded by one occlusion device in a first location and by another occlusion device in a second location proximal to the first location.

The occlusion device in the first location can be either occlusion device 50 or occlusion device 52 if occlusion device 52 is positioned distally from the distal end of outer catheter 4. The occlusion device in the second location can be either occlusion device 5 or the proximally located occlusion device 52 if that device is positioned distally from the distal end of outer catheter 4 and not used to occlude the vessel in the first location. Finally, at 858, an agent is delivered in the region of the blood vessel located between the two expanded occlusion devices at a pressure sufficient to infuse the agent into a localized or semi-localized region of the body.

In the embodiments previously discussed within the Detailed Description section, occlusion of the blood vessel is accomplished using at least two occlusion devices. In contrast to these embodiments, the embodiments described in the following discussion and depicted in FIGS. 13A-B and 15A-C allow occlusion of at least a portion of a blood vessel region with only one occlusion device. It is important to note that even though the nomenclature “inner” and “outer” catheter is retained in discussing these embodiments, each embodiment is capable of operation with only one of either the inner or outer catheters, depending on the needs of the particular application.

FIGS. 13A-C depict an example embodiment where inner catheter 2 is configured to occlude a portion of a blood vessel region by using one axially indented balloon 58 having a side indent 59 in a middle section of cylindrically-shaped balloon 58. FIG. 13A depicts an axial cross section of inner catheter 2 within blood vessel 98 and FIG. 13B depicts a radial cross-section taken along B-B of FIG. 13A. Upon expansion, balloon 58 contacts the inner surface of blood vessel 98 except for the space adjacent to the region 57 corresponding to axial indentation 59. The space adjacent to region 57 is isolated from the remainder of blood vessel 98.

An aperture, or skive, 54 can be placed in region 102 where the indented portion of balloon 58 is coupled with inner catheter 2. Aperture 54 is preferably in fluid communication with the guide wire lumen 17 of inner catheter 2 such that a desired infusion agent can be infused into the isolated region. This allows selective infusion-of a very small region 57 in a directional manner. In other words, only region 57 of vessel 98, isolated in both the axial and radial directions, is exposed to the infusion agent. This is in contrast to the previously described embodiments where infusion takes place at a portion of the blood vessel isolated only in the axial direction, allowing exposure to the infusion agent around the entire circumference of the blood vessel. Valve 56 can also be included for sealing the distal end of inner catheter 2. Balloon 58 is inflated with an inflation medium transmitted through lumen 16 and into balloon 58 via aperture 55. Although not shown, an additional aperture can be placed in region 102. This aperture can provide fluid communication with an additional lumen disposed within inner catheter 2, and can be used to monitor pressure in the isolated space adjacent to indent 59.

FIG. 13C depicts a cross section of vessel 98 having a second vessel 750 branching off. Here, axially indented occlusion balloon 58 can be used to infuse the agent into a semi-localized region from second blood vessel 750. Second blood vessel 750 preferably has a flow restricting configuration 752. One example of this configuration can be found in a vein, venule or other blood vessel, which tapers to a relatively small cross-section that restricts flow to the extent that the desired infusion pressure can be achieved. Another example can be found where a relatively larger vessel branches into multiple smaller vessels or tributaries such that the flow of fluid into the smaller vessels is resisted to a sufficient degree that a desired infusion pressure can be achieved.

For instance, in FIG. 13C, blood vessels 98 and 750 are veins. Vessel 750 has numerous tributaries 751, which can be either smaller veins or venules that flow into vessel 750. The numerous tributaries 751 resist the delivery of an agent from aperture 54 such that continued delivery of the agent raises the fluid pressure within vessels 750 and 751 to a level where. infusion can occur. The infusion can be into a localized region, i.e., occurring through the walls of the vessel 750, or the infusion can be semi-localized, i.e., the infusion pressure can disrupt tributaries 751 to the point at which they will burst or break and thus allow the agent to reach a wider area of surrounding tissue and interstitium.

Because these embodiments in FIGS. 13A-C allow infusion using only the one occlusion balloon 58 located on inner catheter 2, outer catheter 4 is not required and can be optionally eliminated from the catheter system. As noted above, although catheter 2 is referred to as “inner” catheter 2, the term “inner” is retained for purposes of providing clarity and cohesiveness with the embodiments discussed throughout the application. It should be understood that catheter 2 can be used alone, or in combination with outer catheter 4 as desired. Accordingly, use of the term “inner” does not limit the present invention to only embodiments using both the inner and outer catheters.

FIG. 14 depicts infusion method 900, which is one preferred method of infusing an agent into a localized or semi-localized region of the body. Method 900 can be used with any of the catheter embodiments described herein having an axially indented occlusion device. In the preferred embodiment, at 902, catheter 2 having axially indented occlusion device 58 is positioned within a blood vessel. A guide wire can be placed in the vessel prior to positioning of catheter 2, in which case catheter 2 preferably includes valve 56 at the distal end of catheter 2. The guide wire can then be withdrawn from catheter 2 once catheter 2 is in position, at which point valve 56 at least partially closes.

Furthermore, it may be desirable to position catheter 2 such that indented portion 59 is adjacent to an opening in the blood vessel wall that connects the blood vessel containing catheter 2 with a second blood vessel. Preferably, the second blood vessel branches into a plurality of smaller vessels that form a flow restricting configuration that restricts flow to a degree that allows the agent to be delivered at a pressure sufficient to infuse the agent to the semi-localized region.

Next, at 904, occlusion device 58 is expanded such that the blood vessel is occluded by occlusion device 58 and the portion of occlusion device 58 not located in indented area 59 is in contact with the inner surface of the blood vessel. Then, at 906, an agent is delivered in the region of the blood vessel adjacent to indented portion 59 of occlusion device 58 and not in contact with indented portion 59 at a pressure sufficient to infuse the agent into a localized or semi-localized region of the body. Preferably, if valve 56 is used, the pressure exerted by the agent on valve 56 during delivery causes valve 56 to seal.

Similar to the embodiments described with regard to FIGS. 13A-C, the example embodiment depicted in FIGS. 15A-C allows both the creation of an isolated blood vessel region with only one occlusion device and the independent monitoring of pressure during infusion to the isolated region. Here, the sole occlusion device is located on outer catheter 4 and is preferably used to isolate the entire axial length of a blood vessel region and not in a directional manner within a radially limited portion of a blood vessel. This embodiment can be used in applications where the distal portion of the targeted vessel has a flow restricting configuration, limiting any potential downstream escape of the infusion agent. The flow restricting configuration restricts flow to the extent that the desired infusion pressure can be achieved.

FIG. 15A depicts an exterior view of outer catheter 4. FIG. 15B depicts an axial cross-section of outer catheter 4, showing the various interior regions. FIG. 15C depicts a radial cross-section of outer catheter 4, taken along C-C of FIG. 15B. In this embodiment, outer catheter 4 includes outer tubing 72, middle tubing 60 and inner tubing 62. The interior of inner tubing 62 defines lumen 66, which preferably serves as a guide for a guide wire or inner catheter 2. Outer catheter 4 can be used without inner catheter 2 or in combination with inner catheter 2. In the latter case, lumen 66 can be sized to accommodate inner catheter 2, preferably with some room to spare. Inner catheter 2 can be optionally provided with an integrated guide wire (not shown), such as that depicted as element 32 in the embodiments of FIGS. 8A-B and FIG. 9. Lumen 66 is preferably large enough to act as a guide for the guide wire or inner catheter 2 but small enough so that the transition between inner catheter 2 and inner tubing 62 at the distal end of the catheter system does not pose a significant risk of skiving the interior of the blood vessel.

Middle tubing 60 preferably includes a reinforcement, such as metal braiding and the like, for strengthening and stiffening outer catheter 4. Middle tubing 60 and inner tubing 62 are preferably coaxial, with the distal end of middle tubing 60 located proximal to the distal end of inner tubing 62. The annular space between middle tubing 60 and inner tubing 62 defines annular lumen 64. Lumen 64 is preferably used for transmission of the desired infusion agent to the infusion site. Vented cone region 68 is preferably located at the transition between the distal end of middle tubing 60 and inner tubing 62. Here, vented, or slotted, cone region 68 is a transition between the exterior of inner tubing 62 and the distal end of middle tubing 60, which acts as a smooth transition between middle tubing 60 and inner tubing 62 and reduces the risk of skiving the interior of the blood vessel. Also, the distal end of outer tubing 72 is beveled to reduce the risk of skiving. In an alternative embodiment, vented cone region 68 can be coupled with inner tubing 62 and the distal end of outer tubing 72. Cone region 68 also preferably includes one or more openings, vents or slots 69 for infusion, as depicted in FIG. 15A. Vents 69 preferably include smoothed or beveled edges to reduce the risk of skiving.

Outer catheter 4 can include two or more regions of varying stiffness, for example the relatively stiff proximal region 10, softer intermediate region 11 and still softer distal region 12 as well as any curved or bent region for facilitating navigation. Outer catheter 4 can also include pressure monitoring lumen 15 (not shown), if lumen 66 in inner catheter 2 is not used as such. In addition, outer catheter 4 can include proximal occlusion balloon 5, located on outer tubing 72 and preferably in intermediate region 11 shown in FIG. 2A. Balloon 5 is preferably inflated via annular inflation lumen 70, defined by the space between outer tubing 72 and reinforced tubing 60. Balloon 5 can be coupled directly with outer tubing 72 such that outer tubing 72 and balloon 5 form a continuous outer jacket over middle tubing 60. Because this embodiment allows infusion using only the one occlusion balloon 5 located on outer catheter 4, inner catheter 2 is not required and can be optionally eliminated from the catheter system. As noted above, although catheter 4 is referred to as “outer” catheter 4, the term “outer” is retained for purposes of providing clarity and cohesiveness with the embodiments discussed throughout the application. It should be understood that catheter 4 can be used alone, or in combination with inner catheter 2 as desired. Accordingly, use of the term “outer” does not limit the present invention to only embodiments using both the inner and outer catheters.

FIGS. 15A-C depict one example embodiment of outer catheter 4 that can be used to occlude a blood vessel having a flow restricting configuration. It should be noted that in addition to this embodiment, other example embodiments of outer catheter 4 can also be used, such as the embodiment depicted in FIG. 2A. In each of these cases, outer catheter 4 can be used alone or in combination with inner catheter 2.

As discussed above, outer catheter 4 can be used in a blood vessel with a flow restricting configuration at the upstream end of the vessel.

One target application includes the infusion of an agent within the anterior interventricular vein (AIV) of the heart for the purposes of angiogenesis or myogenesis. FIG. 16A depicts human heart 90 including AIV 92, great cardiac vein 94 and coronary sinus 96. The tributaries 97 to AIV 92 constitute a flow restricting configuration 97, which is defined by the numerous veins, small veins and venules that branch into AIV 92. These branches provide enough resistance to fluid flow in a direction opposite blood flow that the desired infusion pressure can be achieved within AIV 92 without the aid of a second occlusion device. In one exemplary method using the catheter system of the present invention, a region of AIV 92 can be substantially isolated by expanding an occlusion device in a location downstream from flow restricting configuration 97. An infusion agent can be delivered to this substantially isolated region of AIV 92 at a desired infusion pressure. The infusion agent is preferably chosen on the basis of qualities that promote angiogenesis or myogenesis.

This region is “substantially” isolated because although some fluid or infusion agent can still pass through flow restricting configuration 97, enough flow is restricted such that the desired pressure for infusion through the wall of AIV 92 can be achieved. While other embodiments refer to isolating a continuous blood vessel segment using multiple occlusion devices, it should be noted that it is not necessary to completely occlude or isolate the vessel for infusion to occur. A blood vessel segment is sufficiently occluded and isolated if the occlusion devices limit the potential collateral escape of the agent enough to allow the desired infusion pressure to be achieved.

Preferably, a catheter system using only one occlusion device, such as outer catheter 4 as depicted in FIGS. 15A-C, is used to perform infusion within AIV 92. However, other outer catheters can also be used, such as outer catheter 4 as depicted in FIGS. 2A-B. Furthermore, this embodiment is not limited to catheter systems using only one catheter and one occlusion device. Catheter systems using two or more catheters and occlusion devices, such as the catheter system depicted in FIGS. 1A-B, can also be used if desired.

FIG. 16B depicts AIV infusion method 1000, which is one preferred method of delivering an infusion agent into a localized or semi-localized region of heart 90 through AIV 92. As described above, the agent can be delivered to a localized region through the blood vessel wall, in this case the wall of AIV 92. The agent can also be delivered to a larger, semi-localized region through the smaller connecting vessels that form a flow restricting configuration, in this case tributaries 97. Delivery to the semi-localized region can be in addition to delivery to the localized region. The actual location where the agent infuses into the surrounding tissue is determined by the thickness of the vessel walls and the fluid pressure in the vessel.

In this preferred embodiment, a guide wire is used in order to properly position outer catheter 4. The guide wire is preferably introduced to coronary sinus 96 at step 1002, either directly or through other surrounding vasculature, and navigated through great cardiac vein 94 into AIV 92 at step 1004. Alternatively, outer catheter 4 can be navigated to through coronary sinus 96 directly, with a curved or bent distal region as depicted in FIGS. 2A-B. Also, a shaped stylet can be placed within lumen 66 and used, either instead of or in combination with the curved region, to facilitate navigation of outer catheter 4 within coronary sinus 96.

Once the guide wire is in place, at step 1006, outer catheter 4 is preferably routed over the guide wire using lumen 66 and positioned within AIV 92 in proximity with the desired infusion site. In one embodiment, navigation is facilitated by the use of radio opaque markers located on outer catheter 4. Once in position, at step 1008 balloon 5 is expanded to occlude AIV 92 and create the substantially isolated region between the expanded occlusion device 5 and flow restricting configuration 97. The infusion agent is then transmitted through lumen 64 and delivered to the substantially isolated region via apertures 69 on vented cone region 68 at step 1010. Delivery of the infusion agent continues in order to increase the fluid pressure within the substantially isolated region to the desired pressure for infusion through the wall of AIV 92 at step 1012 and into a localized region of the body.

The pressure of the infusion agent may be such that infusion occurs into a semi-localized region of heart primarily through the walls of the tributaries defining flow restricting configuration 97 of AIV 92. Once the infusion pressure reaches the desired level, these tributaries become disrupted and porous, and even in some cases burst, thereby allowing the infusion agent to be infused through the walls of the tributaries and into the surrounding tissue at step 1012. While some injury to AIV 92 is preferable in order to allow infusion to occur through the wall of the vessel, fluid pressure within the infusion site is preferably regulated to ensure that only minor injury takes place.

The devices of the present invention may be provided with a pressure regulator to maintain a desired infusion pressure, or to prevent fluid pressure from exceeding a maximum desired pressure in order to maintain a safe environment for the patient. Typically, an infusion pressure at the infusion site of 100-200 mmHg is desired, but greater or lesser pressures may be employed. The pressure regulator can usefully be attached to the input side of the catheter system between the infusion port on the catheter and a syringe or other means used to infuse the desired agent under pressure. The pressure regulator can also be attached to the output side of the catheter system between the pressure feedback and the atmosphere (or a reservoir). The regulator can also be incorporated directly into the syringe or infusion device. The desired pressure at the regulator may be calculated from the desired pressure at the infusion site according to engineering principles well known to those skilled in the art. Several embodiments of pressure regulators useful with the catheter system of the present invention are illustrated in FIG. 17 through FIGS. 25A-B.

FIG. 17 illustrates an example embodiment of an active pressure regulator 100 useful with the catheter system of the present invention. Preferably, this embodiment incorporates the infusion pressure to actively regulate fluid flow. Here, pressure regulator 100 includes housing 302 with lumen 303, valve 304 and infusion feedback chamber 306. The injection device (not shown) is coupled with input 308 to lumen 303 and the catheter system (also not shown) is coupled to output 310 of lumen 303, such that fluid can flow from the injection device, through lumen 303 and into the catheter system.

In this embodiment, a pressure feedback is used to regulate pressure. The pressure feedback is preferably in fluid communication with the isolated blood vessel segment to give a high degree of accuracy in regulation. Here, the pressure feedback is provided by a pressure monitoring lumen, such as pressure monitoring lumen 15, which is coupled to infusion feedback chamber 306 via infusion feedback port 307. The fluid pressure in infusion feedback chamber 306 controls valve 304 and regulates flow accordingly.

Valve 304 includes spool 316 coupled to diaphragms 312 and 314. Spool 316 has through-hole 318 that is preferably aligned with lumen 303 when the spool 316 is centered within the housing 302. Spool 316 is configured to slide laterally within housing 302 such that the misalignment of through-hole 318 can provide increased resistance to fluid flow until it seals lumen 303 completely. Diaphragms 312 and 314 are coupled to spool 316 on opposite sides of lumen 303. Diaphragm 312 is located within infusion feedback chamber 306 and diaphragm 314 is located in an opposing chamber 322. Preferably, both diaphragms 312 and 314 are of equal strength to help maintain spool 316 in an equilibrium position when no other pressures or biases are applied. Here, spool 316 is shown partially misaligned.

When fluid is injected through pressure regulator 100 via lumen 303, the fluid pressure at the target site within the body is fed back to pressure regulator 100 at infusion pressure feedback chamber 306. As the pressure within chamber 306 increases past a predetermined level, a lateral force is exerted on diaphragm 312 causing spool 306 to move laterally away from chamber 306 and begin to seal lumen 303.

The predetermined level is at least partially determined by the bias applied by bias member 320. Bias member 320 is coupled with spool 316 and configured to apply a lateral force in a direction opposite to the fluid pressure force exerted by infusion feedback chamber 306. Spool 316 will only move away from chamber 306 when the fluid pressure force in chamber 306 exceeds the force applied by bias member 320 and diaphragms 312 and 314, in addition to any frictional or gravitational resistances to movement. When the pressure in chamber 306 causes great enough deflection in spool 316, lumen 303 is sealed entirely. Preferably, stop 324 is included to prevent bias member 320 from moving spool 316 too far laterally such that through-hole 318 becomes misaligned with lumen 303 when the fluid pressure in chamber 306 is below the predetermined level. Also, a vent aperture 330 is placed in housing 302 such that the air of other medium within chamber 322 can flow into and out of chamber 322 as required when spool 316 moves.

In this embodiment, bias member 320 is a spring, however, any bias member can be used according to the needs of the application. Because compression coil springs tend to increase in force as the spring is compressed, an increasing amount of force is required to seal lumen 303 entirely in an embodiment that uses a compression coil spring as bias member 320. The threshold point as well as the fluid pressure necessary to seal the lumen 303 entirely can be varied by selecting a bias member 320 with the appropriate compressive and expansive strengths. The bias applied by bias member 320 can also be adjusted by adjustment device 325.

FIGS. 18A-C illustrate a preferred example embodiment of an active pressure regulator 100 useful with the catheter system of the present invention. Preferably, this embodiment incorporates the infusion pressure to actively regulate fluid flow. Here, pressure regulator 100 includes housing 602 with lumens 604 and 606. Preferably, the injection device (not shown) is coupled with a fluid input (not shown) to lumen 604 and the catheter system (not shown) is coupled with a fluid output (not shown) of lumen 604, such that fluid can flow from the injection device, through lumen 604 and into the catheter system. Lumen 604 is preferably composed of a flexible tube, such that lumen 604 can be pinched off, or sealed, by an externally applied force.

Piston 613 is movably disposed within housing 602 such that the motion of piston 613 applies pressure to and seals flexible tube 604. Piston 613 includes bias receiving member 614 and fluid pressure receiving member 615, which are located within cavities 621 and 623, respectively. Members 614 and 615 are coupled together with struts 619, which move within cavities 622. Pinching member 618 is coupled with fluid pressure receiving member 615 and located adjacent to flexible tube 604. Pinching member 618 has a wedge shaped portion configured to contact flexible tube 604 and facilitate the sealing, or pinching off, of lumen 604.

In this embodiment, a pressure feedback is used to actively regulate pressure. The pressure feedback preferably provides fluid communication with the isolated blood vessel segment to give a high degree of accuracy in regulation. Here, the pressure feedback is provided by a pressure monitoring lumen, such as pressure monitoring lumen 15 (not shown), which is coupled to fluid cavity region 628 via fluid input 608. Fluid cavity region 628 is sealed on one side by flexible diaphragm 612. Flexible diaphragm 612 is located adjacent to face 616 of fluid pressure receiving member 615. As the fluid pressure within cavity 628 increases past a predetermined level, diaphragm 612 flexes outward and moves piston 613 in direction 611. The movement of piston 613 causes pinching member 618 to at least partially seal lumen 604. Once the fluid pressure in cavity 628 becomes great enough, lumen 604 is entirely sealed by pinching member 618.

Also illustrated in FIG. 18A is bias member 620. Bias member 620 is configured to apply a bias force to face 617 of bias receiving member 614 in direction 601, which is preferably opposite to direction 611. When the fluid pressure in cavity 628 falls below the predetermined level, bias member 620 pushes piston 613 in direction 601 and at least partially unseals lumen 604, allowing fluid to be injected at a greater rate through lumen 604. Thus, the rate of fluid injection into the catheter system can be actively regulated with the fluid pressure feedback.

The bias force applied by bias member 620 at least partially determines the predetermined level at which piston 613 moves. In a preferred embodiment, the predetermined level where piston 613 begins to move in direction 611 is equal to the bias force applied by bias member 613 plus any frictional and gravitational resistances to movement acting upon piston 613 in direction 601. The bias force applied by bias member 620 can be adjusted by adjustment device 626.

FIGS. 18B and 18C depict side and top views of cap 624, respectively. The removal of cap 624 provides access to the interior of regulator 100. Cap 624 includes key locks 626, which are configured to lock into slots 627 of housing 602. Housing 602 is preferably circular in shape, and cap 624 can be affixed by screwing key lock 626 into slots 627. Cap 624 is preferably composed of a transparent or translucent material such as a polycarbonate, to allow visibility into cavity region 628 to determine if air is present etc. FIG. 18C also shows lumen 606 and the outline of cavity region 628. Diaphragm 612 preferably has a planar shape with a surface area at least partially corresponding to the shape of cavity region 628, thus enabling diaphragm 612 to more adequately receive the fluid pressure within cavity 628.

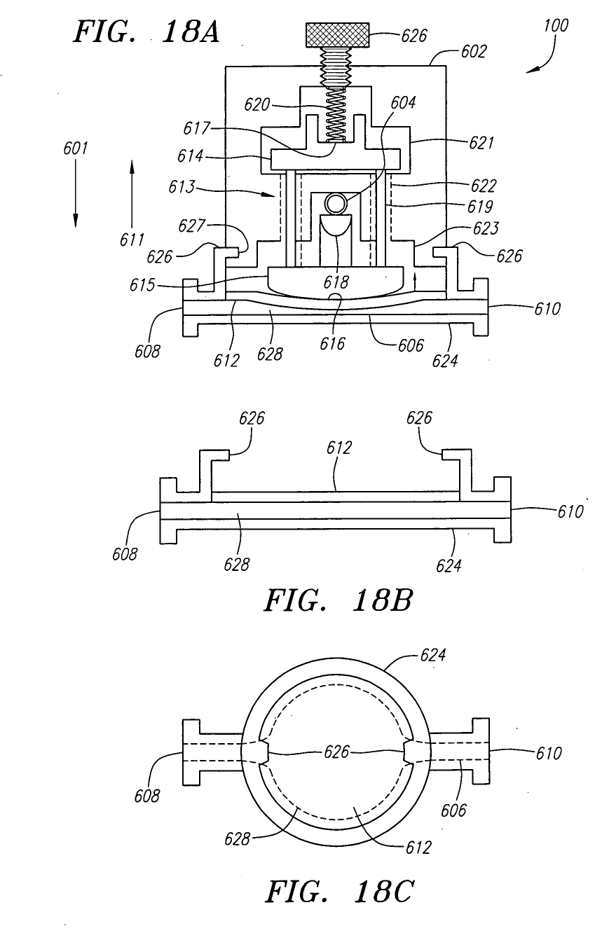

FIGS. 19A-C illustrate another example embodiment of an active pressure regulator 100 useful with the catheter system of the present invention. Again, this embodiment preferably incorporates the infusion pressure to actively regulate fluid flow. FIG. 19A depicts pressure regulator 100 having frame 640, lever arm 658, lumen 642 and inflatable balloon 644. Preferably, the injection device (not shown) is coupled with fluid input 645 to lumen 642 and the catheter system (not shown) is coupled with fluid output 646 of lumen 642, such that fluid can flow from the injection device, through lumen 642 and into the catheter system. Lumen 642 is preferably composed of a flexible tube, such that lumen 642 can be pinched off, or sealed, by a pressure externally applied to tube 642.

In this embodiment, a pressure feedback is used to actively regulate pressure. The pressure feedback preferably provides fluid communication with the isolated blood vessel segment to give a high degree of accuracy in regulation. Here, the pressure feedback is provided by a pressure monitoring lumen, such as pressure monitoring lumen 15 (not shown), which is preferably coupled with inflatable balloon 644 at input 648. Inflatable balloon 644 is located adjacent to lever arm 658, which is pivotably coupled with frame 640 at pivot point 656. Lever arm 658 includes pinching member 660, which extends outward towards flexible tube 642. As the fluid pressure at the isolated blood vessel segment increases past a predetermined level, balloon 644 begins to inflate and rotate lever arm 658 towards flexible tube 642. The rotation of lever arm 658 causes pinching member 660 to pinch off, or seal, flexible tube 642. The degree to which lumen 642 is sealed is directly related to the amount of inflation of balloon 644. Balloon 644 is preferably coupled with lever arm 658 such that as the fluid pressure begins to drop and cause balloon 644 to deflate, lever arm 658 is rotated in the opposite direction and at least partially unseals lumen 642. Thus, the rate of fluid injection into the catheter system can be actively regulated by the fluid pressure feedback provided to balloon 644. Balloon 644 can also include a valve 662 for releasing any undesired any air or gas from balloon 644. In this embodiment, valve 662 is a stop cock. FIG. 19B depicts a side view of regulator 100 taken along B-B of FIG. 19A. Here, frame 640 has a U-shape with side walls 650 and 652, base 654 and cover 661.

The use of lever arm 658 provides mechanical advantages in the mechanical relation between rotation of the arm and sealing lumen 642. By placing the pinching member 660 near pivot point 656 and applying force with balloon 644 along substantially the entire length of arm 658, the amount of leverage needed to seal lumen 642 decreases. Also, the addition of multiple pinching members can increase the sensitivity of regulator 100. FIG. 19C depicts another embodiment of lever arm 658. Here, lever arm 658 includes a second pinching member 662. Pinching member 660 extends outward from lever arm a distance Z₁ and pinching member 662 extends a distance Z₂. In a preferred embodiment, distance Z₁ is greater than distance Z₂ and the two distances are chosen so that the pinching members 660 and 662 will make substantially flush contact with lumen 642 as arm 658 rotates.

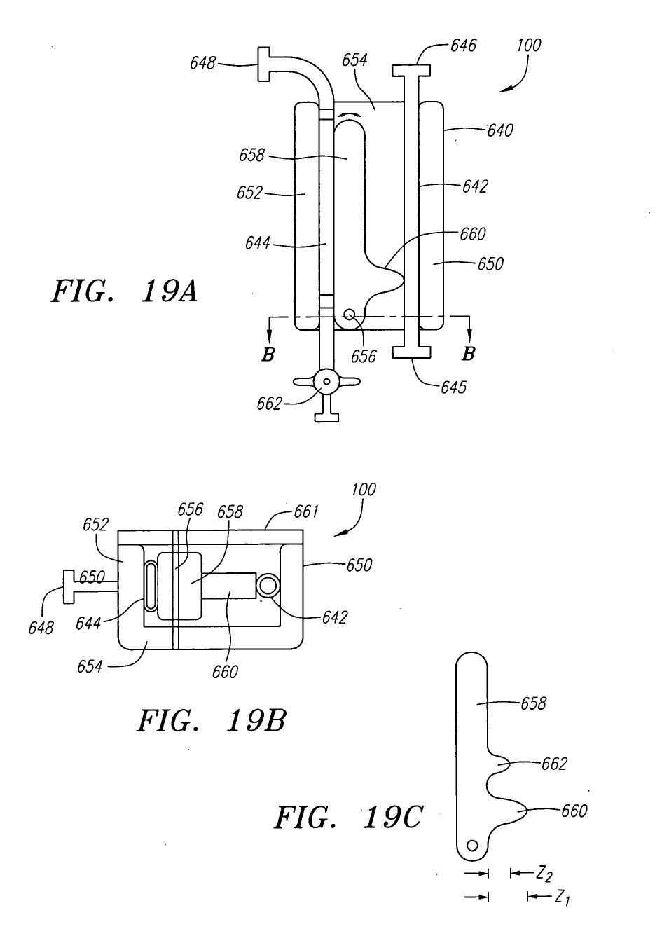

FIGS. 20A-C illustrate another example embodiment of an active pressure regulator 100 useful with the catheter system of the present invention. Preferably, this embodiment incorporates the infusion pressure to actively regulate fluid flow. FIG. 20A depicts pressure regulator 100 having housing 670, lumen 671, piston 674 and fluid cavity 678. Piston 674 is movably disposed within housing 670 and preferably includes body member 675 and pinching member 676. Preferably, the injection device (not shown) is coupled with fluid input 672 to lumen 671 and the catheter system (not shown) is coupled with fluid output 673, such that fluid can flow from the injection device, through lumen 671 and into the catheter system. Lumen 671 is preferably composed of a flexible tube, such that lumen 671 can be sealed by an externally applied pressure. Flexible tube 671 is preferably located within hollow channel 685.

In this embodiment, a pressure feedback is used to actively regulate pressure. The pressure feedback is preferably in fluid communication with the isolated blood vessel segment to give a high degree of accuracy in regulation. Here, the pressure feedback is provided by a pressure monitoring lumen, such as pressure monitoring lumen 15 (not shown), which is preferably coupled with cavity 678 via fluid input 679. Flexible diaphragm 680 preferably forms a wall of cavity 678, located adjacent to face 677 of body member 675. Body member 675 is located on a first side of flexible tube 671. Pinching member 676 extends from body member 675 to a second side of lumen 671 that is opposite the first side such that lumen 671 is located between pinching member 676 and body 675.

As the fluid pressure within cavity 678 increases past a predetermined level, diaphragm 680 flexes outward and moves piston 674 in direction 690. The movement of piston 674 causes pinching member 676 to at least partially seal lumen 671. Once the fluid pressure in cavity 678 becomes great enough, lumen 671 is entirely sealed by pinching member 676.

Also illustrated in FIG. 20A is bias member 681 and groove 693. Bias member 681 is configured to apply a bias force to face 694 of body member 675 in direction 691, which is preferably opposite to direction 690. Bias member is coupled between body member 675 and cover member 682. When the fluid pressure in cavity 678 falls below the predetermined level, bias member 681 moves piston 674 in direction 691 and at least partially unseals lumen 671, allowing fluid to be injected at a greater rate through lumen 671. Thus, the rate of fluid injection into the catheter system can be actively regulated by the fluid pressure feedback provided to cavity 678.

Groove 693 in housing 670 is provided to allow the movement of piston 674 in directions 690 and 691. FIG. 20B is a perspective view that illustrates the motion of piston 674. Here, only piston 674 and flexible tube 671 are shown the movement of body member 675 in direction 690 causes pinching member 676 to at least partially seal lumen 671. Conversely, movement of body member 675 in direction 691 causes pinching member 676 to at least partially seal lumen 671.

The bias force applied by bias member 681 can at least partially determine the predetermined level at which piston 674 moves. In a preferred embodiment, the predetermined level where piston 674 begins to move in direction 690 is equal to the bias force applied by bias member 681 plus any frictional and gravitational resistances to movement acting upon piston 674 in direction 691.

FIG. 20C depicts a top view of regulator 100 without cover member 682. Here, bleed port 683 is coupled with valve 684, which in this embodiment is a bleed screw. Valve 684 allows an adjustable amount of fluid to bleed from cavity 678 and increases the fluid pressure necessary to flex diaphragm 680. Thus, the adjustment of valve 684 also adjusts the predetermined level at which piston 674 will begin to regulate flow through lumen 671.

In the above discussion, various embodiments are presented that use a member to pinch a flexible lumen in order to restrict the flow of fluid through that lumen. It should be noted that in certain applications, the fluid passing through the lumen can be an agent comprised of a biological cells. In these applications, the design and construction of the various pinching members and flexible lumens should take into account the risk of pinching the flexible lumen in such a way that the cells are ruptured.

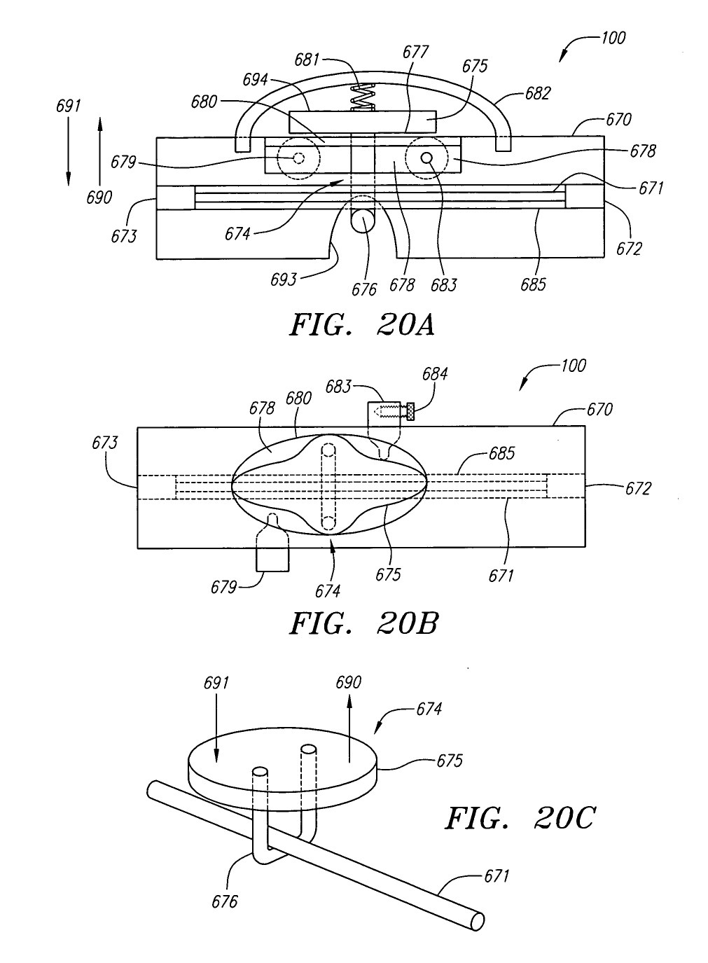

FIG. 21 depicts yet another embodiment of an active pressure regulator 100. Here, housing 950 includes a flexible tube 956 coupled with fluid input 964 and fluid output 966, which are preferably coupled with an injection device and the catheter system, respectively. Inflatable balloon 952 is located within housing 950 and has an input coupled with fluid input 970, which is in turn preferably coupled with a pressure monitoring lumen. The pressure monitoring lumen is preferably in fluid communication with the site of infusion and provides a fluid pressure feedback therefrom. Balloon 952 can also have an output coupled with fluid output 972 of housing 950. Fluid output 972 can be coupled with a valve, such as a stopcock and the like, and used to bleed any undesired air or inflation medium from within balloon 952.

Also included within housing 950 is cam 954. Cam 954 is movably disposed within housing 950 in cavity 962. Cam 954 is coupled with bias element 960, which is in turn coupled with housing 950 within cavity 962. Cam 954 has two opposing sides 951 and 953. Side 951 is located adjacent to balloon 952. Opposite side 953 has pinching member 955 extending outwards towards flexible tube 956. Flexible tube 956 can optionally include an inner jacket 957 and a harder, outer jacket 958, in which case an opening 959 in outer jacket 958 is preferably provided and aligned with pinching member 955 to allow pinching member 955 to contact inner jacket 957.

Bias element 960 applies a bias force to maintain cam 954 in position. When the fluid pressure in balloon 952 reaches a predetermined level, balloon 952 begins to inflate and move cam 954 and pinching member 955 towards flexible tube 956 and cause pinching member 955 to at least partially seal tube 956. As the fluid pressure within balloon 952 increases, balloon 952 continues to expand and cause pinching member 955 to increasingly seal flexible tube 956. The predetermined level at which balloon 952 begins to inflate can be dependent on numerous factors including the material elasticity of balloon 952, the bias force applied by bias member 960, frictional forces and the like.

Furthermore, the amount of movement or deflection of cam 954 necessary to completely seal tube 956 can be adjusted with adjustment device 976. Adjustment device 976 preferably adjusts plate 974 both towards and away from flexible tube 956 in order to increase and decrease, respectively, the amount of movement needed by cam 954 to seal tube 956. In this embodiment, adjustment device 976 is a screw knob that screwably adjusts plate 974. Adjustment device 976 can optionally include a visible indicator to indicate the fluid pressure necessary to seal tube 956 at each position of device 976.

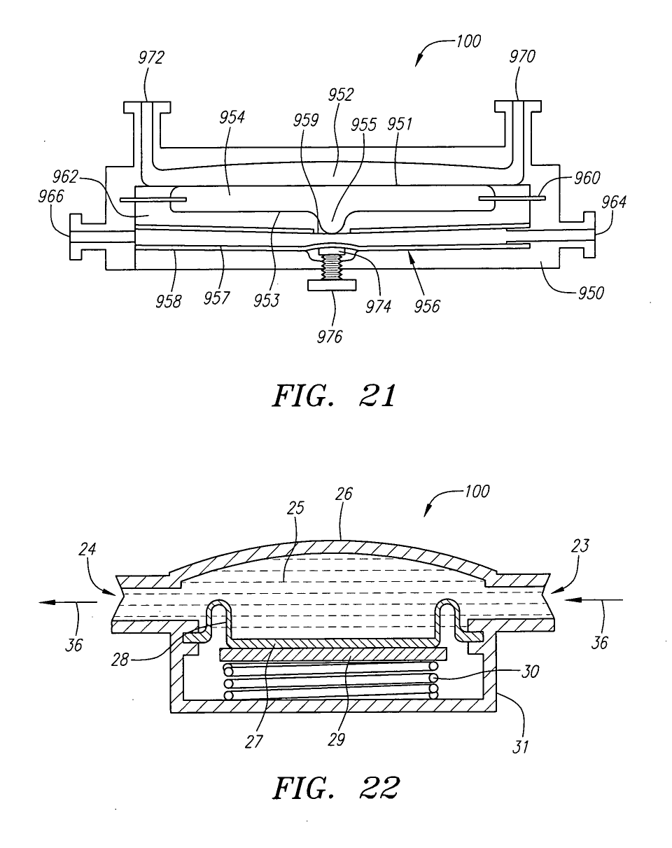

FIG. 22 depicts one example embodiment of a passive pressure regulator 100. Here, the direction of fluid flow is indicated by the arrows 36 shown adjacent the inlet 23 and the outlet 24 of the pressure regulator 100. The infusion fluid passes through cavity 25 in the pressure regulator which is formed by wall 26 and diaphragm 27 and flexible element 28. In a preferred embodiment, the diaphragm is circular in configuration.

Plate 29 is coupled to spring element 30 which may be a coil, leaf or other type of spring. A coil spring is illustrated. The spring is also coupled to the shell 31 of the pressure regulator. Pressure is regulated by the counter forces of the pressure of the fluid in cavity 25 and the pressure exerted by spring 30. When the pressure in cavity 25 exceeds the desired pressure, diaphragm 27 will be brought into contact with plate 29 and the spring force in spring 30 will counter undesired over pressurization in cavity 25.

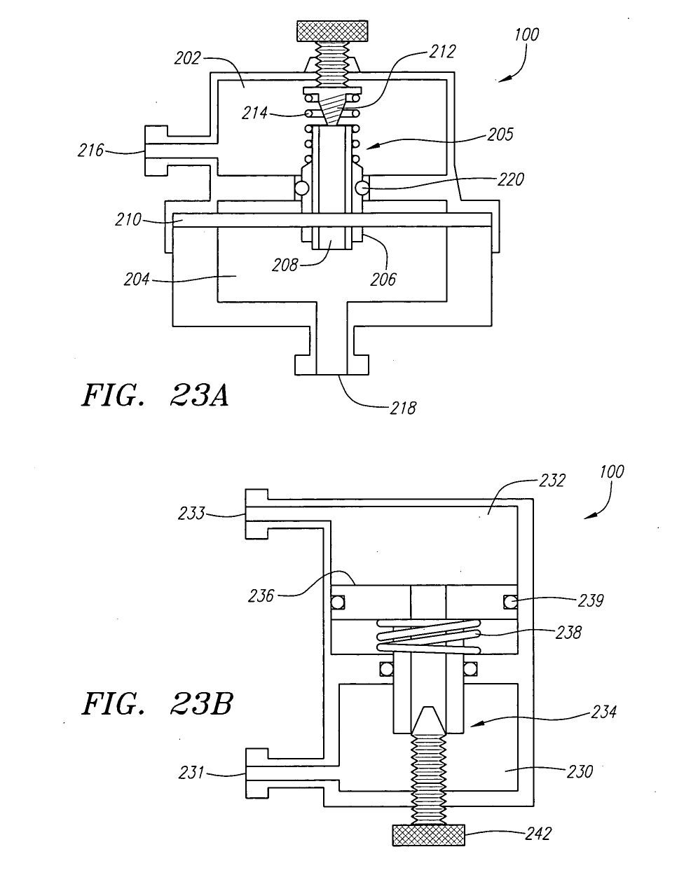

FIG. 23A illustrates another example embodiment of a passive pressure regulator 100 useful with the catheter system of the present invention. In this embodiment, pressure regulator 100 includes two chambers, inlet chamber 202 and outlet chamber 204, which are located opposite to each other. Valve 205 is located between the two chambers and regulates resistance to fluid flow between chambers 202 and 204. Valve 205 includes deflectable piston 206 with through-hole 208 that allows fluid to pass. Valve 205 further includes plunger 212 aligned with through-hole 208 and configured to seal through-hole 208 when piston 206 is deflected towards plunger 212. In this embodiment, valve 205 is a variable resistance needle valve. However, valve 205 can be any valve capable of sealing through-hole 208. Preferably, piston 206 is coupled with diaphragm 210 that allows piston 206 to deflect into each chamber 202 and 204 according to the respective pressures exerted on piston 206. Bias member 214, in this embodiment a spring, is further coupled between piston 206 and plunger 212 and applies a bias, or pressure, forcing piston 206 and plunger 212 apart and into an open position. Diaphragm 210 also biases piston 206 to remain in the open position.

To inject an infusion agent in a pressure regulated manner, an injection device such as a syringe (not shown) can be coupled with fluid input 216 while the catheter system (not shown) can be coupled to fluid output 218. The injection device can then be used to inject the infusion agent into the catheter system through pressure regulator 100. In this embodiment, the position of piston 206 is determined by the pressure exerted by bias member 214, diaphragm 210 and the fluid pressure in outlet chamber 204, which is primarily a result of the amount of force exerted by the injection device to inject fluid into outlet chamber 204 by way of through-hole 208.

Once the fluid pressure in outlet chamber 204 becomes equal to or greater than the pressure of bias member 214, a threshold point is reached, and piston 206 deflects towards plunger 212. As piston 206 approaches plunger 212, the resistance to fluid flow increases. Once the pressure applied by diaphragm 210 becomes great enough, valve 205 closes and prevents further fluid flow into the catheter system.

The amount of travel of piston 206, the deflectability of diaphragm 210, the compressive and expansive pressures applicable by bias member 214, the location of plunger 212 as well as the resistance to deflection incurred by any seal 220 located on piston 206, are all variables that should be considered in the design of pressure regulator 100. Preferably, the resistance to movement created by seal 220 and the positioning of piston 206 are not substantial relative to the pressure applied by bias member 214 and diaphragm 210. Pressure regulator 100 can be further configured with an adjustment device for allowing valve 205 to seal at varying fluid pressures. Here, the depth of plunger 212 is adjustable by coupling plunger 212 with inlet chamber 202 via a rotatable threaded knob.

FIG. 23B illustrates another embodiment of a passive pressure regulator 100, configured without a diaphragm. Here, pressure regulator includes two chambers located opposite to each other, specifically inlet chamber 232 with fluid input 233 and outlet chamber 230 with fluid output 231. Regulator 100 also includes valve 234. Valve 234 includes deflectable piston 236 having through-hole 237, bias member 238 and plunger 240. Bias member 238 biases piston 236 towards a closed position. In this case, the infusion agent can be injected with an injection device (not shown) coupled directly into a catheter system (not shown). The catheter preferably includes a pressure feedback, preferably as a pressure monitoring lumen, which is coupled to fluid input 233. Although this embodiment includes a pressure feedback, the pressure is regulated passively because pressure regulation does not occur at the syringe or other injection device at the input to the catheter system.

When the pressure feedback fluid pressure acting on piston 236 becomes greater that the counteracting pressure exerted by bias member 238, the threshold point is reached and piston 236 is deflected away from plunger 240. Because valve 234 is a needle valve in this embodiment, the pressure is reduced as piston 236 moves away from plunger 240. Also, as in the above embodiment, pressure regulator 100 can optionally include adjustment device 242 for adjusting the point at which valve 234 seals. Also shown is seal 239 coupled with piston 236. Seal 239 provides some resistance to movement by piston 236, preferably this resistance is not substantial relative to the bias applied by bias member 238. Piston 236 could be replaced by a diaphragm as shown in FIG. 23A above.

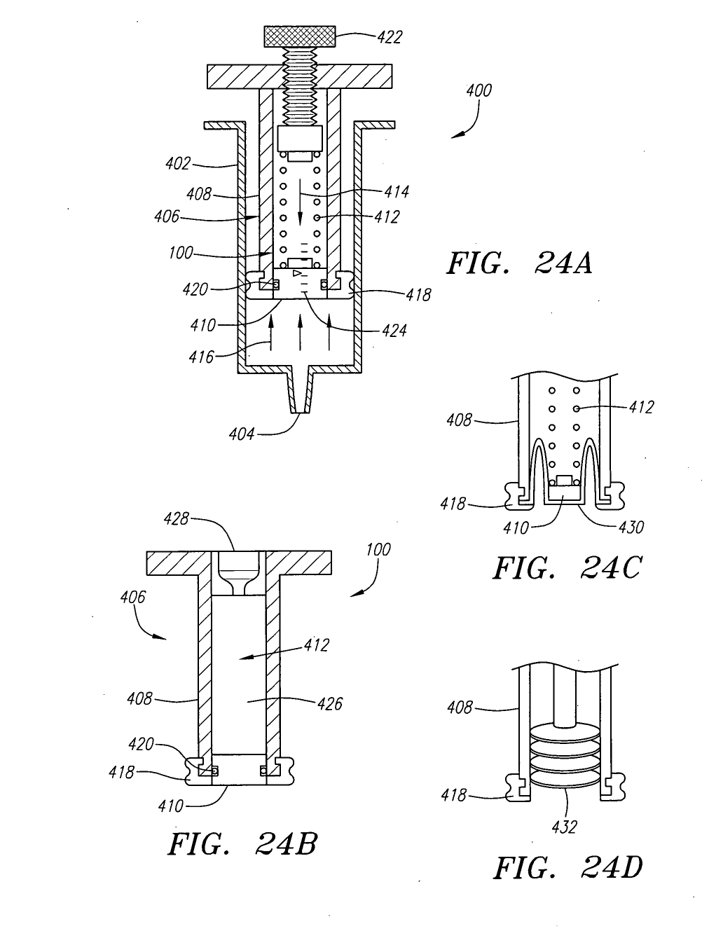

FIGS. 24A-D and 25A-B illustrate additional example embodiments of a passive pressure regulator 100 useful with the catheter system of the present invention. As opposed to the previous embodiments, which regulate fluid pressure by directly adjusting the allowable flow, these pressure regulators 100 regulate pressure by accumulating excess fluid to maintain the pressure at an acceptable level.

In the embodiment illustrated in FIGS. 24A-D, passive pressure regulator 100 is incorporated directly into injection device 400. Injection device 400 can be any device configured to inject an infusion agent into the catheter system and, in this embodiment, injection device 400 is a syringe. Syringe 400 includes housing 402 for holding a fluid an outputting the fluid through fluid output 404. Syringe 400 also includes plunger 406 slidably coupled with the housing 402, plunger 406 configured to force the fluid out of fluid output 404 in a conventional manner known to those of skill in the art.

In the preferred embodiment, pressure regulator 100 is incorporated within plunger 406. Here, pressure regulator 100 includes slidable piston 410 housed within the plunger body 408. Piston 410 is coupled with bias member 412 that is configured to apply pressure to piston 410 in direction 414, thereby maintaining piston 410 in an extended state as illustrated in FIG. 24A. As plunger 406 is depressed, the fluid in housing 402 exerts a fluid pressure force in all directions within the housing, including a force on piston 410 in direction 416. When the fluid pressure force exceeds the force applied by bias member 412, the device has reached a threshold point and piston 410 retracts into plunger body 408. This retraction increases the volume of the housing 402 and counteracts the depression of plunger 406, thereby accumulating excess fluid within housing 402. This relieves the fluid pressure at fluid output 404 and regulates the maximum fluid pressure attainable by the injection device 400. Once depression of the plunger 406 slows and the fluid pressure falls to a low enough level, bias member 412 returns piston 410 to the extended state.

As noted above, in embodiments using a compression coil spring for bias member 412, the compressive force applied by spring 412 in direction 414 varies based on the extent to which spring 412 is compressed. Accordingly, because of the different levels of compression, the point where spring 412 begins to return piston 410 to the extended state will generally be greater than the threshold point required for piston 410 to initially retract.

Also, if plunger 406 is depressed very rapidly, the fluid pressure within housing 402 will spike, or increase rapidly and cause piston 410 to retract. The fluid pressure in housing 402 will then decay as the depression of plunger 406 slows, or as piston 410 returns to the extended state. There is a time delay for this pressure spike to travel through the catheter system to the infusion site, and the infusion site may experience only a reduced pressure spike or even none at all. This embodiment can therefore provide insulation to pressure spikes as well as regulation of the fluid pressure at the infusion site.

In this embodiment, injection device 400 also includes seal 418 at the base of plunger body 408 for sealing off housing 402. Piston 410 includes O-ring seal 420 for sealing the inner cavity of plunger body 408 from fluid within housing 402. Also, injection device 400 can optionally include adjustment device 422 for adjusting the threshold point. In this embodiment, adjustment device 422 is a threaded knob screwably coupled with plunger body 408. As the knob 422 is screwed into plunger body 408, bias member 412 is compressed and the threshold point becomes larger. However, in order to reduce the complexity of injection device 400 in actual applications, pressure regulator 100 can be configured to provide the proper compressive forces without the need for additional adjustment device 422.

In addition, injection device 400 can include direct pressure scale 424 located on piston 410 and aligned with a marking on piston 410. This pressure scale 424 can be used to determine the fluid pressure being applied either within housing 402 or within the isolated blood vessel region.

FIG. 24B illustrates another example embodiment of piston 410 where bias member 412 is an air spring. Air spring 412 includes cavity 426, which can be filled with a gas, such as air. As opposed to a compression coil spring, air spring 412 provides a constant compressive force in direction 414 as piston 410 retracts. This embodiment also includes pressure relief valve 428 for releasing the air within cavity 426 as piston 410 retracts. If desired, a secondary mechanism can be used to return piston 410 to the extended state. Also, air and a coil spring can be combined together to provide the compressive force in piston 410.

FIG. 24C illustrates another example embodiment of piston 410 where O-ring seal 420 is replaced with roller diaphragm 430. Roller diaphragm 430 is composed of a deformable material such as rubber, plastic or the like, and allows piston 410 to extend or retract within plunger body 408. Roller diaphragm 430 preferably reduces the frictional force between piston 410 and plunger body 408, while at the same time sealing piston 410 from fluid in housing 402. The reduced frictional force gives piston 410 a quicker response, i.e., it allows piston 410 to respond to smaller changes in fluid pressure in less time than an embodiment with O-ring 420. Because enough room between piston 410 and plunger body 408 must exist for unrestricted movement of roller diaphragm 430, a reduction in the radial size of piston 410, or an increase in the radial size of plunger body 408, may be required.

FIG. 24D illustrates another example embodiment where piston 410 and O-ring seal 420 are replaced with bellows 432. The operation of bellows 432 is similar to the roller diaphragm 430, where a reduced frictional force, such as the stick/slip friction, with plunger body 408 lowers the threshold level and allows a quicker response to fluid pressure. However, in this embodiment bias member 412 can also be eliminated and bellows 432 can act as a piston, seal and bias member together. Bellows 432 is preferably composed of material that can adequately transmit force according to the needs of the embodiment. Such materials can include soft plastic, metal and rubber. Furthermore, bellows 432 can be combined with a coil or air spring or other bias member to transmit force.

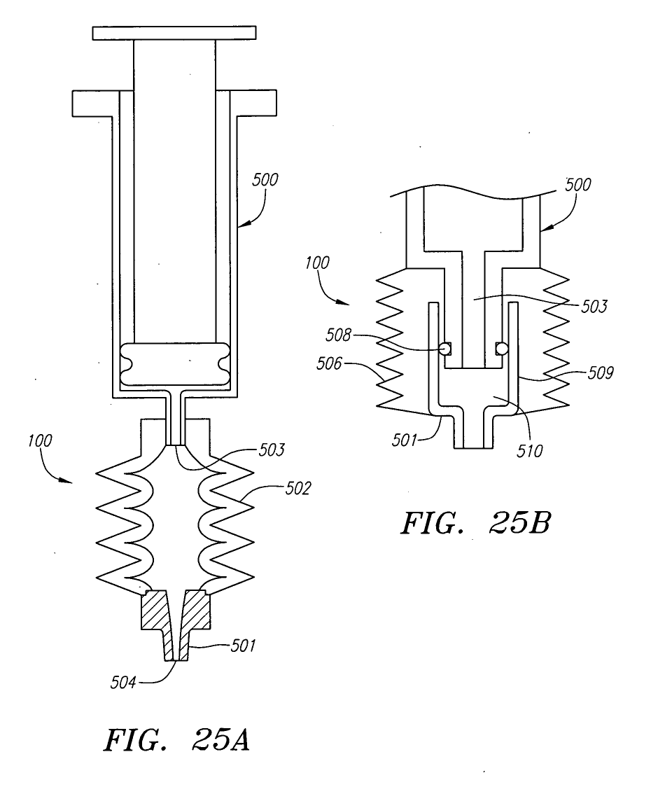

FIGS. 25A-B illustrate additional accumulator-type embodiments of passive pressure regulator 100. In FIG. 25A, injection device 500 is coupled to output port 501 by expandable bellows 502. The expandable bellows 502 expands and accumulates fluid when the output pressure between output port 501 and injection device output 503 exceeds a threshold point. When the fluid pressure falls to a low enough level, bellows 502 begins to compress and force the excess fluid through output port 501 and into the catheter system. Like a compression coil spring, bellows 502 exhibits a varying compressive force depending on the level of compression or expansion that bellows 502 is at. Accordingly, the threshold point can be adjusted by adjusting the type of bellows 502 used, including the composition, size and number of individual bellows within pressure regulator 100.

FIG. 25B illustrates a similar embodiment to the one in FIG. 25A, except here bellows 502 is replaced with extension spring 506 and O-ring seal 508. Output port 501 is coupled to injection device output 503 by extension spring 506. When the fluid pressure at injection device output 503 exceeds the threshold point of spring 506, output port 501 begins to extend from the retracted state shown in FIG. 25B. As output port 501 extends, sleeve 509 slides extends from device output 503 and increases the volume of cavity 510, defined by sleeve 509, device output 503 and output port 501. Cavity 510 accumulates the excess fluid to maintain the fluid pressure at an acceptable level. O-ring seal 508 seals the junction between fluid output 503 and sleeve 509. As in the other embodiments, the threshold point of the regulator can be adjusted by adjusting the type of spring 506 used, or by adjusting an adjustment device, such as a threaded sleeve, to adjust the initial tension force. Once the fluid pressure in cavity 510 recedes, extension spring 506 can then compress and output the excess fluid into the catheter system.

As stated above, one preferred use of the catheter system of the present invention is with the methods of Yock et al. U.S. Pat. No. 6,346,098 which are centered on localized and semi-localized delivery of an infusion agent through the wall of a blood vessel. Because blood vessel structure can vary widely, the catheter systems and methods used to isolate and seal the vessel must vary accordingly. For instance, because the physiologic venous pressures are much lower than arterial pressures, veins tend to be more compliant and have much thinner walls than arteries. Therefore, an expanded occlusion device will tend to distend, or stretch, the walls of the vein to a greater degree than would occur within an artery. Also, venous walls exert less resistance to expansion, and therefore the force exerted by the venous wall against the occlusion device is less than the force that would be exerted by an arterial wall. The lesser force allows fluids to pass between the occlusion device and the venous wall more easily and makes occlusion more difficult, especially when infusion pressures in the range of 100-200 mmHg, and even up to 400 mmHg are sought. Simply increasing the diameter of an expanded occlusion device may lead to injury and rupture of the vein.

In order to adequately occlude and isolate a venous blood vessel region with balloons 3 and 5, the balloons 3 and 5 preferably achieve nominal diameters of between 0.25 and 6 atmospheres (atm) of pressure, either fluid or air pressure, where higher pressures result in higher occlusion forces against the walls. The nominal diameter can vary according to the needs of the individual application. Experimental studies have shown that nominal diameters substantially in the range of 6-8 millimeters (mm), such as 6.0, 6.5 and 8.0 mm, all can provide optimal trade-off between occlusion force and expansion of the venous walls. The nominal diameter will vary according to the size of the targeted blood vessel region.

In addition, the balloons are preferably configured to expand in diameter by 1-2 mm with the addition of 2-3 atm of fluid pressure. This allows the use of one balloon 3 or 5 in multiple venous diameters. If balloons 3 and 5 are too non-compliant (e.g., nylon, Polyethylene, Polyethylene Terephthalate) they may not be adjustable for vessel size as indicated above. If the balloons are too compliant (e.g. silicone rubber, latex, low-durometer Polyurethane) they may not provide enough occlusive force at their nominal diameters. Experimentation and simulation results have found that high durometer elastomeric materials, such as 55D Polyurethane, provide an optimal or near-optimal trade-off between compliant and non-compliant materials.

In addition to the material composition and fluid pressure factors, the balloon geometry should also be considered for proper occlusion. Preferably, balloons 3 and 5 have a cylindrical shape instead of a spherical shape in order to maximize the sealing area, or working length, with the wall. By increasing the working length, a higher infusion pressure can be achieved without creating dangerously high pressures to the venous walls. The working length can be chosen based on the needs of the application, such as the need to balance the required infusion pressure with the desire to achieve the smallest balloon diameter possible to facilitate handling. Preferably, the working length of balloons 3 and 5 are in the range of 1-2 centimeters (cm) although longer or shorter lengths can be used as desired.

The catheter system described herein can be furnished to a user, such as a medical professional, in the form of a kit. The kit preferably includes inner catheter 2, outer catheter 4, pressure regulator 100, an agent and instructions for use. The kit can include any number of guide wires, such as 0.014″, 0.018″ and 0.035″ guide wires and the like. The kit can also include radio opaque dye or markers for facilitating navigation of the catheter or guide wire. The radio opaque dye can be optionally mixed with the agent if desired and if it does not significantly inhibit the therapeutic or diagnostic qualities of the agent.

The kit can be further customized for a desired application. For instance, in a preferred embodiment the catheter system is inserted into the AIV of a patient to treat angiogenesis or myogenesis. A kit customized for use in this application can also include one or more stylets or dilators, configured to aid in advancing the catheter system in the patient's vasculature. In the case where outer catheter 4 is shaped or curved to facilitate navigation within the vasculature, the dilator can be used to straighten out outer catheter 4.

If either inner catheter 2 or outer catheter 4 require additional shaping to facilitate navigation, a stylet can be used. One or more stylets can be provided for different anatomies, each stylet being customized to facilitate navigation into a target vessel. The stylet can be configured according to the needs of the application, by adjusting the stiffness, shape and/or composition of the stylet. In one example embodiment, the stylet is flexible enough to straighten while being inserted into the body, yet is stiff enough to maintain the desired shape while within inner catheter 2 or outer catheter 4 and the vasculature. The stylet can also be malleable such that it can be configured directly to the anatomy prior to use. This stylet can be hollow such that it can be advanced over a guide wire and can also be optionally composed of a shape memory material. In one example embodiment, a teflon-coated non-annealed stylet is used. However, other configurations and hardnesses can also be used.

Also, pressure regulator 100 can be customized to regulate the pressure in a user friendly manner, for instance without the need for adjustment of the threshold point or without the need for monitoring a pressure scale by the user. Furthermore, the occlusion devices can be sized and configured for the target blood vessels within the coronary sinus. Integrated guide wire 32 can optionally be provided in combination with inner catheter 2. Kits can be customized for any application using the present catheter system, and the kits are not limited solely to the treatment of angiogenesis or myogenesis and are also not limited to use within the coronary sinus.

In the foregoing specification, the invention has been described with reference to specific embodiments thereof. It will, however, be evident that various modifications and changes may be made thereto without departing from the broader spirit and scope of the invention. For example, the reader is to understand that the specific ordering and combination of process actions described herein is merely illustrative, and the invention can be performed using different or additional process actions, or a different combination or ordering of process actions. For example, this invention is particularly suited for applications involving the infusion of an agent in an isolated blood vessel, but can be used in any application involving blood vessel isolation. As a further example, each feature of one embodiment can be mixed and matched with other features shown in other embodiments. Features and processes known to those of ordinary skill in the art of catheter systems may similarly be incorporated as desired. Additionally and obviously, features may be added or subtracted as desired. Accordingly, the invention is not to be restricted except in light of the attached claims and their equivalents.

1. A catheter system for creating an isolated blood vessel region comprising:
a first catheter and a second catheter, each having a proximal and a distal end, the first catheter having a first expandable occlusion device associated therewith and the second catheter having a second expandable occlusion device associated therewith, the first catheter being adapted to expand the first occlusion device distally of the second occlusion device on the second catheter,
the first catheter being slidably housed within a first lumen in the second catheter such that the distance between the first and second occlusion devices may be varied,
the occlusion devices being expandable to engage a wall of a blood vessel thereby substantially isolating an interior region of a desired extent between the first and second occlusion devices, wherein the second catheter has a relatively stiff proximal region and a softer distal region.

2. The catheter system of claim 1, wherein the expandable occlusion device is located on the relatively stiff proximal region.

3. The catheter system of claim 1, wherein the expandable occlusion device is located on the softer distal region.

4. The catheter system of claim 1, wherein the second catheter has an intermediate region located between the distal and proximal regions, the intermediate region being softer than the proximal region and stiffer than the distal region.

5. The catheter system of claim 4, wherein the second expandable occlusion device is located on the intermediate region.

6. The catheter system of claim 4, wherein the expandable occlusion device is located on the relatively stiff proximal region.

7. The catheter system of claim 4, wherein the expandable occlusion device is located on the softer distal region.

8. The catheter system of claim 1, wherein the second catheter has a second lumen disposed within, the second lumen in communication with the second occlusion device and configured to transmit an inflation medium to the second occlusion device.

9. The catheter system of claim 1, wherein the second catheter has a pre-formed curve to facilitate navigation within a blood vessel.

10. The catheter system of claim 9, wherein the first lumen of the second catheter is configured to slidably receive a dilator configured to substantially straighten the pre-formed curve.

11. The catheter system of claim 1, wherein the first lumen of the second catheter has an open distal end.

12. The catheter system of claim 1, wherein the second catheter comprises and inner tubing and an outer tubing positioned in a coaxial configuration and defining a second lumen therebetween.

13. The catheter system of claim 12, wherein the second lumen is in communication with the second occlusion device and configured to transmit an inflation medium to the second occlusion device.

14. The catheter system of claim 1, wherein the first catheter has a first lumen disposed within and configured to slidably receive a guide wire.

15. The catheter system of claim 14, wherein the first catheter has a second lumen disposed within, the second lumen in communication with the first occlusion device and configured to transmit an inflation medium to the first occlusion device.

16. The catheter system of claim 15, wherein the first catheter has a third lumen disposed within, the third lumen having an open distal end located proximal to the first occlusion device.

17. The catheter system of claim 16, further comprising a pressure regulator coupled with an open proximal end of the third lumen.

18. The catheter system of claim 14, wherein the first lumen of the first catheter is configured to slidably receive a stylet.

19. The catheter system of claim 1, further comprising a pressure regulator coupled between an injection device and an open proximal end of the first lumen of the second catheter.

20. The catheter system of claim 1, wherein the first lumen of the second catheter is configured to slidably receive a stylet.

21. A catheter system for creating an isolated blood vessel region comprising:
a first catheter and a second catheter, each having a proximal and a distal end,
the first catheter having a first balloon associated therewith, and a second catheter having a second balloon associated therewith, wherein the first catheter is adapted to expand the first balloon distally of the second balloon on the second catheter,
the first catheter being slidably housed within a first lumen in the second catheter such that the distance between the first and second balloons may be varied,
the balloons being expandable to engage a wall of a blood vessel thereby substantially isolating an interior region of a desired extent between the first and second balloons, wherein the balloons are cylindrically shaped and configured to achieve a nominal diameter at substantially between 0.25 and 6 atmospheres of pressure.

22. The catheter system of claim 21, wherein at least one of the balloons has a nominal diameter of substantially between 6 and 8 millimeters.

23. The catheter system of claim 22, wherein at least one of the balloons has a nominal diameter of 6 millimeters.

24. The catheter system of claim 22, wherein at least one of the balloons has a nominal diameter of 6.5 millimeters.

25. The catheter system of claim 22, wherein at least one of the balloons has a nominal diameter of 8 millimeters.

26. The catheter system of claim 21, wherein at least one of the balloons is configured to be expandable from the nominal diameter by an additional 1-2 millimeters with the application of an additional 1-2 atmospheres of pressure.

27. The catheter system of claim 21, wherein the balloons are composed of 55D polyurethane.

28. The catheter system of claim 21, wherein the balloons have a working length of between 1-2 centimeters.

29. A catheter system for creating an isolated blood vessel region comprising:
an inner catheter and an outer catheter, each having a proximal and a distal end, the inner catheter having a first and a second expandable occlusion device associated therewith and the outer catheter having a third expandable occlusion device associated therewith, the inner catheter being adapted to expand the first occlusion device distally of the second occlusion device on the inner catheter and the third occlusion device on the outer catheter,
the inner catheter being slidably housed within a first lumen in the outer catheter such that the distance between the first and third occlusion devices may be varied,
wherein the first and second occlusion devices are expandable to engage a wall of a blood vessel thereby substantially isolating a fixed interior region between the first and second occlusion devices, and wherein the first and third occlusion devices are expandable to engage a wall of a blood vessel thereby substantially isolating an interior region of a desired extent between the first and third occlusion devices.

30. The catheter system of claim 29, wherein the inner catheter further comprises an aperture located between the first and second occlusion devices, the aperture allowing infusion from an infusion lumen located within the inner catheter.

31. A catheter system for creating an isolated blood vessel region comprising:
a first catheter comprising a proximal and a distal end, an inner lumen and a first occlusion device, wherein the first catheter is configured to expand the first occlusion device; and
a guide wire comprising a proximal and a distal end and housed within the inner lumen of the first catheter, wherein the guide wire is integrated with the distal region of the first catheter.

32. The catheter system of claim 31, further comprising:
a second catheter comprising a proximal and a distal end, a second occlusion device and an inner lumen configured to slidably receive the first catheter such that the distance between the first and second occlusion devices may be varied, wherein the second catheter is configured to expand the second occlusion device proximally of the first occlusion device on the first catheter; and
further wherein the occlusion devices are expandable to engage a wall of a blood vessel thereby substantially isolating an interior region of a desired extent between the first and second occlusion devices.

33. The catheter system of claim 31, wherein the first catheter further comprises a second occlusion device.

34. The catheter system of claim 31, further comprising an atraumatic tip covering the distal end of the guide wire.

35. The catheter system of claim 31, wherein the guide wire is integrated with the distal end of the first catheter at a distal tip of the first catheter.

36. The catheter system of claim 35, wherein the distal tip is curved to facilitate navigation within a body.

37. The catheter system of claim 35, wherein the distal tip is closed and covers the distal end of the guide wire.

38. The catheter system of claim 35, wherein the distal tip is composed of a radio opaque material and is coupled to the distal end of the first catheter.

39. The catheter system of claim 38, wherein the distal tip is thermally bonded to the distal end of the first catheter.

40. The catheter system of claim 35, wherein the guide wire is integrated with the distal tip over substantially the entire length of the distal tip.

41. The catheter system of claim 35, wherein the first occlusion device is located proximal to the distal tip.

42. The catheter system of claim 41, wherein the first occlusion device is a balloon integrally coupled with the first catheter.

43. The catheter system of claim 42, wherein the first balloon and first catheter form a continuous outer jacket over the portion of the guide wire housed within the first lumen.

44. The catheter system of claim 32, wherein the second occlusion device is a balloon integrally coupled with the second catheter.

45. A catheter system for creating an isolated blood vessel region comprising:
a catheter having a proximal and a distal end and an expandable occlusion device associated therewith, the expandable occlusion device having a cylindrical shape and an axial indent in a middle section of the device, wherein a portion of the indentation is coupled with a region of the catheter, and wherein an aperture is located in the region where the device and catheter are coupled together, the aperture providing fluid communication with an inner lumen of the catheter.

46. The catheter system of claim 45, further comprising a second aperture in the region where the device and catheter are coupled together, the aperture providing fluid communication with a second inner lumen of the catheter.

47. The catheter system of claim 45, wherein the inner lumen of the catheter is configured to slidably receive a guide wire and wherein a distal end of the inner lumen has a valve configured to allow the guide wire to pass therethrough.

48. The catheter system of claim 47, wherein the valve is configured to at least partially close upon removal of the guide wire.

49. The catheter system of claim 48, wherein the catheter is configured to deliver an agent from the inner lumen through the aperture, and further wherein the valve is configured such that the pressure exerted by the agent within the lumen causes the valve to seal.

50. A catheter system for creating an isolated blood vessel region comprising:
a catheter having a proximal and a distal end, the catheter having an expandable occlusion device associated therewith and being adapted to expand the occlusion device, the occlusion device being expandable to engage a wall of a blood vessel thereby substantially isolating an interior region of a desired extent between the occlusion device and a flow restricting configuration of the vessel, wherein the catheter has a relatively stiff proximal region and a softer distal region.

51. The system of claim 50, wherein the first expandable occlusion device is located on the relatively stiff proximal region.

52. The system of claim 51, wherein the first catheter has an intermediate region located between the proximal and distal regions, the intermediate region being softer than the proximal region and stiffer than the distal region.

53. The system of claim 52, wherein the first expandable occlusion device is located on the intermediate region.

54. The system of claim 52, wherein the first expandable occlusion device is located on the relatively stiff proximal region.

55. The catheter of claim 50, further comprising a pressure monitoring lumen configured to measure the fluid pressure in the isolated blood vessel region.

56. A catheter system for creating an isolated blood vessel region comprising:
a catheter having a distal and a proximal end, comprising:
an outer tubing with an expandable occlusion device associated therewith, the outer tubing having a distal end;
a middle tubing having a distal end and extending within the outer tubing, the space between the middle tubing and the outer tubing defining a first lumen; and
an inner tubing having a third distal end and coupled with the middle tubing and extending within the middle tubing such that the distal end of the inner tubing is located distal to the distal end of the middle tubing, the space between the middle tubing and the inner tubing defining a second lumen and the space within the inner tubing defining a third lumen,
a cone-shaped element coupled with the exterior of the inner tubing and having an opening in fluid communication with the second lumen.

57. The catheter system of claim 56, wherein the occlusion device is an occlusion balloon and the first lumen is configured to pass an inflation medium to the occlusion balloon.

58. The catheter system of claim 56, wherein the third lumen is further configured to slidably receive a second catheter.

59. The catheter system of claim 56, wherein the third lumen is further configured to slidably receive a guide wire.

60. The catheter system of claim 56, wherein the cone-shaped element is coupled between the exterior of the inner tubing and the distal end of the middle tubing.

61. The catheter system of claim 56, wherein the cone-shaped element is coupled between the exterior of the inner tubing and the distal end of the outer tubing.

62. The catheter system of claim 56, further comprising an inner catheter having a second occlusion balloon associated therewith, wherein the third lumen is configured to slidably receive the inner catheter such that the distance between the first and second occlusion balloons may be varied.

63. The catheter system of claim 56, wherein the middle tubing is reinforced.

64. A catheter system for isolating a segment of a blood vessel, comprising:
a catheter having a distal end and a lumen configured to slidably receive a guide wire, the distal end having a valve configured to allow the guide wire to pass therethrough and at least partially close upon removal of the guide wire;
wherein the catheter has an aperture for delivering an agent from the lumen therethrough, and further wherein the valve is configured such that the pressure exerted by the agent within the lumen causes the valve to seal.

65. The catheter system of claim 64, wherein the catheter has a first expandable occlusion device associated therewith.

66. The catheter system of claim 65, wherein the catheter has a second expandable occlusion device associated therewith.

67. The catheter system of claim 66, wherein the catheter has a second lumen in communication with the first and second occlusion devices and configured to pass an inflation medium to the occlusion devices.

68. A method of infusing an agent into a localized or semi-localized region of the body, comprising:
positioning an inner catheter and an outer catheter within a blood vessel, wherein the inner and outer catheters each have an occlusion device associated therewith, and the outer catheter has an open distal end and is configured to slidably receive the inner catheter;
positioning the inner catheter occlusion device distally from the distal end of the outer catheter;
expanding the occlusion devices such that the blood vessel is occluded by the inner catheter occlusion device in a first location and by the outer catheter occlusion device in a second location proximal to the first location;
delivering an agent in the region of the blood vessel located between the two expanded occlusion devices at a pressure sufficient to infuse the agent into a region of the body external to the blood vessel.

69. The method of claim 68, wherein the agent is delivered through the blood vessel wall and into a localized region of the body.

70. The method of claim 68, wherein the blood vessel has a connecting side vessel in the region between the two occlusion devices, the side vessel connecting with a plurality of smaller vessels that form a flow restricting configuration, and wherein the agent is delivered through at least one of the smaller vessels and into a semi-localized region of the body.

71. The method of claim 68, wherein the outer catheter occlusion device is expanded prior to the inner catheter occlusion device.

72. The method of claim 68, wherein the inner catheter occlusion device is expanded prior to the outer catheter occlusion device.

73. The method of claim 68, further comprising using a guide wire previously positioned within the blood vessel to facilitate positioning of the inner and outer catheters within the blood vessel.

74. The method of claim 68, wherein positioning the inner and outer catheter within the blood vessel comprises:
positioning the inner catheter within the blood vessel; and
slidably advancing the outer catheter over the inner catheter and into position within the blood vessel.

75. The method of claim 74, further comprising:
advancing a guide wire into the blood vessel prior to positioning the inner catheter; and
slidably advancing the inner catheter over the guide wire and into position within the blood vessel.

76. The method of claim 74, further comprising positioning the inner catheter within the blood vessel with a stylet.

77. The method of claim 74, further comprising positioning the inner catheter within the blood vessel by advancing the inner catheter through the blood vessel using a guide wire integrated with the inner catheter.

78. The method of claim 68, wherein positioning the inner and outer catheter within the blood vessel comprises:
positioning the outer catheter within the blood vessel; and
slidably advancing the inner catheter within the outer catheter and into position within the blood vessel.

79. The method of claim 78, further comprising:
advancing a guide wire into the blood vessel prior to positioning the outer catheter; and
slidably advancing the outer catheter over the guide wire and into position within the blood vessel.

80. The method of claim 78, further comprising advancing the outer catheter into position within the blood vessel.

81. The method of claim 80, further comprising advancing the outer catheter into position within the blood vessel with a stylet located within the outer catheter.

82. The method of claim 80, wherein a distal region of the outer catheter has a pre-formed curve to facilitate advancement of the outer catheter within the blood vessel.

83. The method of claim 82, further comprising straightening the pre-formed curve of the outer catheter with a dilator to facilitate advancement of the outer catheter.

84. The method of claim 68, wherein the agent has characteristics that promote angiogenesis.

85. The method of claim 68, wherein the agent has characteristics that promote myogenesis.

86. The method of claim 68, further comprising monitoring the pressure during delivery of the agent.

87. The method of claim 87, further comprising regulating the pressure during delivery of the agent with a pressure regulator.

88. The method of claim 87, wherein the pressure is regulated passively.

89. The method of claim 87, wherein the pressure is regulated actively with a pressure monitoring lumen providing a fluid pressure feedback from the region of the blood vessel between the occlusion devices to the pressure regulator.

90. The method of claim 68, wherein the blood vessel is a vein located within the heart.

91. The method of claim 90, wherein the blood vessel is the anterior interventricular vein (AIV).

92. The method of claim 68, further comprising delivering radio opaque dye to the blood vessel prior to delivering the agent.

93. The method of claim 68, wherein the agent is delivered through the open distal end of the outer catheter.

94. The method of claim 68, wherein the agent is delivered through an opening in the inner catheter located between the two occlusion devices.

95. A method of infusing an agent into a localized or semi-localized region of the body, comprising:
advancing a guide wire into the blood vessel;
slidably advancing a catheter over the guide wire using a lumen within the catheter;
positioning the catheter within a blood vessel, the catheter having a first and a second occlusion device associated therewith, wherein the first occlusion device is located distally from the second occlusion device;
expanding the occlusion devices such that the blood vessel is occluded by the first occlusion device in a first location and by the second occlusion device in a second location proximal to the first location;
delivering an agent from the lumen and into the region of the blood vessel located between the two expanded occlusion devices at a pressure sufficient to infuse the agent into a region of the body external to the blood vessel.

96. The method of claim 95, wherein the agent is delivered through the blood vessel wall and into a localized region of the body.

97. The method of claim 95, wherein the blood vessel has a connecting side vessel in the region between the two occlusion devices, the side vessel connecting with a plurality of smaller vessels that form a flow restricting configuration, and wherein the agent is delivered through at least one of the smaller vessels and into a semi-localized region of the body.

98. The method of claim 95, wherein the agent is delivered from the lumen within the catheter through an opening in the catheter located between the two occlusion devices.

99. The method of claim 95, wherein the occlusion devices are balloons.

100. The method of claim 99, further comprising expanding the balloons by passing an inflation medium through a lumen in communication with each of the two occlusion devices.

101. The method of claim 95, further comprising withdrawing the guide wire prior to delivering the agent.

102. The method of claim 101, wherein a distal end of the catheter includes a valve configured to allow the guide wire to pass therethrough.

103. The method of claim 102, further comprising at least partially closing the valve upon withdrawal of the guide wire.

104. The method of claim 102, wherein the valve is configured such that the pressure exerted by the agent on the valve during delivery causes the valve to seal.

105. The method of claim 95, further comprising delivering a radio opaque substance to monitor the infusion of the agent into the region of the body.

106. The method of claim 95, further comprising delivering a radio opaque substance with the agent to monitor the infusion of the agent into the region of the body.

107. A method of infusing an agent into a localized or semi-localized region of the body, comprising:
positioning an inner catheter and an outer catheter within a blood vessel, the outer catheter having an open distal end and configured to slidably receive the inner catheter, the outer catheter having an occlusion device associated therewith and the inner catheter having a first and a second occlusion device associated therewith with the first occlusion device located distally from the second occlusion device;
positioning at least one of the inner catheter occlusion devices distally from the distal end of the outer catheter;
expanding at least two of the occlusion devices such that the blood vessel is occluded by one occlusion device in a first location and by another occlusion device in a second location proximal to the first location, wherein the occlusion device in the first location is either the first inner catheter occlusion device or the second inner catheter occlusion device if the second device is positioned distally from the distal end of the outer catheter, and wherein the occlusion device in the second location is either the outer catheter occlusion device or the proximally located inner catheter occlusion device if that device is positioned distally from the distal end of the outer catheter and not used to occlude the vessel in the first location;
delivering an agent in the region of the blood vessel located between the at least two expanded occlusion devices at a pressure sufficient to infuse the agent into a localized region of the body.

108. The method of claim 107, wherein the agent is delivered through the blood vessel wall and into a localized region of the body.

109. The method of claim 107, wherein the blood vessel has a connecting side vessel in the region between the at least two occlusion devices, the side vessel connecting with a plurality of smaller vessels that form a flow restricting configuration, and wherein the agent is delivered through at least one of the smaller vessels and into a semi-localized region of the body.

110. The method of claim 107, wherein the distally located first inner catheter occlusion device is occludes the vessel in the first location and the outer catheter occlusion device occludes the vessel in the second location.

111. The method of claim 110, wherein the proximally located second inner catheter occlusion device remains unexpanded during delivery of the agent.

112. The method of claim 110, wherein the agent is delivered through the open distal end of the outer catheter.

113. The method of claim 110, further comprising monitoring the pressure in the region of the blood vessel located between the at least two expanded occlusion devices during delivery of the agent.

114. The method of claim 107, wherein the distally located first inner catheter occlusion device is occludes the vessel in the first location and the proximally located second inner catheter occlusion device occludes the vessel in the second location.

115. The method of claim 114, wherein the outer catheter occlusion device is left unexpanded during delivery of the agent.

116. The method of claim 115, wherein the agent is delivered through an aperture in the inner catheter located between the two occlusion devices associated with the inner catheter.

117. The method of claim 114, further comprising monitoring the pressure in the region of the blood vessel located between the at least two expanded occlusion devices during delivery of the agent.

118. A kit for providing a catheter system for use in the delivery of an infusion agent to an isolated blood vessel region, comprising:
a first catheter having a proximal end, a distal end and a first expandable occlusion device associated therewith, and a second catheter having a proximal end and a distal end and a second expandable occlusion device associated therewith, wherein the first catheter is configured to expand the first occlusion device distally of the second occlusion device on the second catheter, the first catheter being slidably housed within a first lumen in the second catheter such that the distance between the first and second occlusion devices may be varied, the occlusion devices being expandable to engage a wall of a blood vessel thereby substantially isolating an interior region of a desired extent between the first and second occlusion devices, wherein the first lumen is configured to deliver an agent to the isolated interior region; and
a pressure regulator configured to regulate the fluid pressure of the agent.

119. The kit of claim 118, further comprising an agent.

120. The kit of claim 118, further comprising a stylet.

121. The kit of claim 120, wherein the second catheter has a pre-formed curve.

122. The kit of claim 121, further comprising a dilator.

123. The kit of claim 118, further comprising a guide wire comprising a proximal and a distal end and housable within the first lumen, wherein the distal end of the guide wire is integrated with the distal end of the first catheter.

124. The kit of claim 118, wherein the second catheter has a relatively stiff proximal region and a softer distal region.

125. The kit of claim 124, wherein the second expandable occlusion device is located on the relatively stiff proximal region.

126. The kit of claim 124, wherein the second expandable occlusion device is located on the softer distal region.

127. The kit of claim 124, wherein the first catheter has an intermediate region located between the proximal and distal regions, the intermediate region being softer than the proximal region and stiffer than the distal region.

128. The kit of claim 127, wherein the second expandable occlusion device is located on the intermediate region.

129. The kit of claim 127, wherein the second expandable occlusion device is located on the relatively stiff proximal region.

130. The kit of claim 127, wherein the second expandable occlusion device is located on the softer distal region.

131. A method of delivering an infusion agent to a semi-localized region of the body, comprising:
advancing a catheter into a blood vessel using a guide wire integrated with the catheter;
positioning an occlusion device located on the catheter within the blood vessel, the blood vessel connecting with a plurality of smaller vessels located distal to the occlusion device, wherein the plurality of smaller vessels are in a flow restricting configuration;
expanding the occlusion device to occlude the blood vessel and substantially isolate a region of the blood vessel defined by the occlusion device and the distally located flow restricting configuration;
delivering an agent to the substantially isolated region of the blood vessel at a pressure sufficient to infuse the agent through at least one of the plurality of smaller vessels and into the semi-localized region of the body.

132. The method of claim 131, wherein the blood vessel comprises the anterior interventricular vein (AIV).

133. The method of claim 132, wherein the smaller vessels are tributaries.

134. The method of claim 133, wherein positioning the catheter comprises advancing the catheter through a coronary sinus and a great cardiac vein and into the AIV.

135. The method of claim 133, wherein the pressure is sufficient to infuse the agent through the vessel wall of the AIV and into the semi-localized region.

136. The method of claim 135, wherein the plurality of tributaries includes at least one venule.

137. The method of claim 131, wherein the catheter comprises a closed distal tip that covers the distal end of the guide wire.

138. The method of claim 131, further comprising positioning a guide wire in proximity with the infusion site prior to positioning the catheter.

139. The method of claim 138, wherein the step of positioning the catheter comprises routing the catheter over the guide wire using a lumen disposed within the catheter.

140. A method of infusing an agent into a localized or semi-localized region of the body, comprising:
positioning a catheter within a blood vessel, the catheter having an occlusion device associated therewith, wherein the occlusion device has an axially indented portion in a middle section of the device;
expanding the occlusion device to occlude the blood vessel and create an isolated space in the blood vessel adjacent to the axially indented portion of the occlusion device;
delivering an agent in the space of the blood vessel adjacent to the indented portion of the occlusion device and not in contact with the indented portion of the occlusion device at a pressure sufficient to infuse the agent into a region of the body expanding the occlusion device.

141. The method of claim 140, wherein the agent is delivered from a lumen within the catheter through a first opening in the catheter located in a region of the catheter where the indent is coupled with the catheter.

142. The method of claim 141, wherein the occlusion device is a balloon.

143. The method of claim 141, further comprising monitoring the pressure of the agent during delivery through a second opening in the catheter located in the region of the catheter where the indent contacts the catheter.

144. The method of claim 141, further comprising positioning the catheter within the blood vessel by advancing the catheter through the blood vessel using a guide wire integrated with the catheter.

145. The method of claim 141, further comprising:
advancing a guide wire into the blood vessel prior to positioning the catheter; and
slidably advancing the catheter over the guide wire and into position within the blood vessel, the catheter configured to slidably receive the guide wire, within the lumen.

146. The method of claim 145, further comprising withdrawing the guide wire prior to delivering the agent.

147. The method of claim 146, wherein a distal end of the catheter includes a valve configured to allow the guide wire to pass therethrough.

148. The method of claim 147, further comprising at least partially closing the valve upon withdrawal of the guide wire.

149. The method of claim 148, wherein the valve is configured such that the pressure exerted by the agent on the valve during delivery causes the valve to seal.

150. The method of claim 140, further comprising delivering an agent in the space of the blood vessel adjacent to the indented portion of the occlusion device and not in contact with the indented portion of the occlusion device at a pressure sufficient to infuse the agent through the blood vessel wall and into a localized region of the body.

151. The method of claim 140, further comprising positioning the catheter within the blood vessel such that the indented portion of the occlusion device is adjacent to an opening in the blood vessel wall that connects the blood vessel containing the catheter with a second blood vessel, wherein the second blood vessel branches into a plurality of smaller vessels that form a flow restricting configuration that restricts flow to a degree that allows the agent to be delivered at a pressure sufficient to infuse the agent to a semi-localized region.

152. The method of claim 151, wherein the pressure is sufficient to disrupt the smaller vessels and allow the agent to infuse through at least one of the smaller vessels and into the semi-localized region.

153. The method of claim 151, wherein the pressure is sufficient to disrupt the wall of the second blood vessel and allow the agent to infuse through at least a portion of the wall of the second vessel and into the semi-localized region.

154. A method of infusing an agent into a localized or semi-localized region of the body of a patient, comprising:
expanding an occlusion device located on a catheter within a blood vessel to isolate a portion of the blood vessel;
delivering an agent from a first lumen within the catheter to the isolated portion of the vessel at a pressure sufficient to infuse the agent through the wall of the blood vessel;
monitoring the fluid pressure of the agent in the isolated portion of the blood vessel with a second lumen; and
regulating the fluid pressure of the agent to maintain the fluid pressure below a desired level.

155. The method of claim 154, wherein the desired level is chosen to avoid injury to a patient.

156. The method of claim 155, wherein the pressure is regulated actively.

157. The method of claim 156, wherein the pressure is regulated with a pressure regulator coupled with the first and second lumens.

158. The method of claim 154, further comprising expanding a second occlusion device distally from the first to isolate a portion of the blood vessel defined by the first and second occlusion devices.

159. An injection system for pressure regulated injection of a fluid into an isolated blood vessel region having a pressure regulator comprising:
a housing having a lumen located between a fluid input and a fluid output;
a spool movably disposed within the housing, the spool having a first end, a second end and a through-hole alignable with the lumen such that fluid can pass through the lumen only when the through-hole is at least partially aligned with the lumen; and
a fluid pressure feedback coupled with the spool and configured to monitor the fluid pressure at the isolated blood vessel region and move the spool at least partially out of alignment with the lumen when the fluid pressure at the blood vessel exceeds a predetermined level.

160. The system of claim 159, further comprising a bias member coupled to the first end of the spool and configured to apply a bias force to maintain the spool in alignment with the lumen, wherein the predetermined level is at least partially determined by the bias force applied by the bias member.

161. The system of claim 159, wherein the first and second ends of the spool are coupled to a first and a second diaphragm, respectively, wherein each diaphragm is configured to keep the through-hole in at least partial alignment with the lumen when the fluid pressure is below the threshold point.

162. The system of claim 161, wherein the fluid pressure feedback is coupled to a fluid pressure feedback chamber and wherein the second diaphragm is located in the fluid pressure feedback chamber such that the second diaphragm moves the spool when the force of the fluid pressure in the feedback chamber on the second diaphragm exceeds the threshold point.

163. The system of claim 162, wherein the threshold point is determined in part by the resistance to moving the spool provided by the first diaphragm, second diaphragm and the bias member.

164. An injection system having a pressure regulator for pressure regulated injection of a fluid into an isolated blood vessel region, comprising:
a housing having a first lumen and a second lumen, wherein the first lumen is at least partially composed of a flexible tube and has a fluid input coupled with an injection device and a fluid output coupled with a catheter system, and wherein the second lumen has a fluid input coupled with a pressure monitoring lumen and is at least partially composed of a flexible diaphragm; and
a piston movably disposed within the housing, the piston comprising a first face adjacent to the diaphragm and a pinching member adjacent the flexible tube;
wherein the flexible diaphragm is configured to flex in a first direction when the fluid pressure in the second lumen exceeds a predetermined level, the diaphragm configured to contact the first face and move the piston in the first direction and at least partially seal the tube with the pinching member.

165. The system of claim 164, further comprising:
a bias member in contact with a second face of the piston and configured to apply a bias force to the second face in a second direction opposite the first direction, wherein the predetermined level is at least partially determined by the bias force applied by the bias member.

166. The system of claim 165, wherein the bias member is configured to move the piston in the second direction when the fluid pressure in the second lumen falls below the predetermined level and at least partially unseal the flexible tube with the pinching member.

167. The system of claim 166, wherein the bias force applied by the bias member is adjustable.

168. The system of claim 166, wherein the bias member is a spring having a first end in contact with the second face of the piston and a second end in contact with the base of an adjustable screw such that adjustment of the screw adjusts the bias applied by the bias member.

169. The system of claim 164, wherein the pinching member is wedge-shaped where the member contacts the tube.

170. The system of claim 164, wherein the second lumen has a sealable outlet configured to allow the release of air from the second lumen.

171. The system of claim 170, wherein the sealable outlet is a stopcock.

172. The system of claim 170, wherein the first lumen includes a fluid cavity region located adjacent to the diaphragm.

173. The system of claim 172, wherein the diaphragm has a planar shape with a surface area at least partially corresponding to the cavity region.

174. An injection system having a pressure regulator for pressure regulated injection of a fluid into an isolated blood vessel region, comprising:
a lumen having a fluid input coupled with an injection device and a fluid output coupled with a catheter system, wherein the lumen is mounted in a frame and is at least partially composed of a flexible tube;
an inflatable balloon having an input coupled with a pressure monitoring lumen, the balloon being configured to inflate when the fluid pressure in the pressure monitoring lumen increases past a predetermined level; and
a lever arm pivotably coupled with the frame at a pivot point and located between the balloon and the flexible tube, the lever arm having a first pinching member extending outwards from the lever arm towards the flexible tube;
wherein the balloon, lever arm and tube are located within the frame such that the inflation of the balloon causes the pinching member to rotate towards the flexible tube and at least partially seal the flexible tube.

175. The system of claim 174, wherein the frame comprises a base and a first and second side wall, the balloon located adjacent to and in contact with the first side wall and the flexible tube located adjacent to and in contact with the second sidewall.

176. The system of claim 174, wherein the balloon is coupled with the lever arm.

177. The system of claim 176, wherein the balloon is configured to deflate when the fluid pressure in the pressure monitoring lumen decreases past a predetermined level and rotate the pinching member away from the flexible tube and at least partially unseal the flexible tube.

178. The system of claim 174, wherein the lever arm has a second pinching member located between the first pinching member and the pivot point and extending outwards from the lever arm towards the flexible tube.

179. The system of claim 175, wherein the length of the second pinching member is greater than the length of the first pinching member, and wherein the lengths of each pinching member are such that the two pinching members make substantially flush contact with the flexible tube when the lever arm rotates towards the flexible tube.

180. An injection system having a pressure regulator for pressure regulated injection of a fluid into an isolated blood vessel region, comprising:
a housing having a lumen having a fluid input coupled with an injection device and a fluid output coupled with a catheter system, wherein the lumen is at least partially composed of a flexible tube;
a piston movably disposed within the housing, the piston having a body located on a first side of the tube and a pinching member extending from the body to a second side of the tube opposite the first side such that the tube is located between the body and the pinching member, wherein the body has a first face;
a fluid cavity located in the housing between the piston body and the flexible tube and having a fluid input coupled with a pressure monitoring lumen and a first wall comprising a flexible diaphragm located adjacent to the first face of the piston body;
wherein the diaphragm is configured to flex in a first direction when the fluid pressure in the cavity exceeds a predetermined amount and contact the first face of the piston body and move the piston in the first direction and at least partially seal the tube with the pinching member.

181. The system of claim 180, wherein the housing further comprises a cover member covering the piston body.

182. The system of claim 181, further comprising:
a bias member located between the cover member and the piston body and in contact with the cover member and a second face of the piston and configured to apply a bias force to the second face in a second direction opposite the first direction, wherein the predetermined level is at least partially determined by the bias force applied by the bias member.

183. The system of claim 182, wherein the bias member is configured to move the piston in the second direction when the fluid pressure in the second lumen falls below the predetermined level and at least partially unseal the flexible tube with the pinching member.

184. The system of claim 183, wherein the predetermined level is adjustable.

185. The system of claim 184, further comprising:
a bleed port in the fluid cavity; and
a valve coupled with the port and configured to adjustably seal the port;
wherein the adjustment of the valve adjusts the amount of fluid that can bleed from the port.

186. The system of claim 185, wherein the valve is a bleed screw.

187. The system of claim 182, wherein the pinching member is U-shaped and surrounds the tube.

188. An injection system having a pressure regulator for pressure regulated injection of a fluid into an isolated blood vessel region, comprising:
a housing having a first lumen at least partially composed of a flexible tube, a first and second fluid input and a first fluid output, wherein the first fluid input is coupled with an injection device, the second fluid input is coupled with a pressure monitoring lumen and the first fluid output is coupled with a catheter system;
an inflatable balloon having an input coupled with the second fluid input;
a bias member coupled between the housing and a cam movably disposed within the housing between the flexible tube and the balloon;
the cam having a first side in contact with the inflatable balloon and a second side opposite to the first, wherein a pinching member extends outwards from the second side towards the flexible tube;
wherein the balloon, pinching member and tube are located within the housing such that the inflation of the balloon causes the pinching member to move towards the flexible tube and at least partially seal the flexible tube.

189. The system of claim 188, wherein the housing has a second fluid output coupled with a valve and wherein the balloon has an output coupled with the second fluid output.

190. The system of claim 188, wherein the flexible tube comprises an inner jacket and a harder outer jacket, the harder, outer jacket having an opening aligned with the pinching member to allow the pinching member to contact the inner jacket.

191. The system of claim 190, further comprising an adjustable plate located adjacent to the flexible tube and opposite the opening in the outer jacket of the tube, the plate configured to adjustably move towards the flexible tube and at least partially seal the flexible tube.

192. The system of claim 188, wherein the bias member is a planar spring.

193. An injection system for pressure controlled injection of a fluid into an isolated blood vessel region, comprising:
a housing having a first chamber coupled to a fluid input and a second chamber coupled to a fluid output;
a deflectable piston having a through-hole and located between the two chambers such that fluid can pass between the chambers by way of the through-hole, wherein the deflectable piston is biased towards an open position; and
a valve, located in one of the two chambers and aligned with the through-hole such that the valve can at least partially seal the through-hole, wherein the deflectable piston deflects towards the valve and away from the open position when the fluid pressure in the chamber opposite the valve exceeds the bias on the deflectable piston.

194. The system of claim 193, wherein the piston is coupled with a diaphragm located in the chamber opposite the valve, the diaphragm configured to bias the piston towards the open position and to deflect the piston towards the valve when the fluid pressure in the chamber opposite the valve exceeds the bias on the deflectable piston.

195. The system of claim 194, further comprising a bias member configured to bias the piston towards the open position.

196. The system of claim 195, wherein the valve is located in the first chamber.

197. The system of claim 194, wherein the valve is a needle valve configured to increasingly seal the through-hole as the degree of deflection of the piston increases.

198. The system of claim 197, wherein the needle valve is adjustably coupled to the housing and adjustable to vary the fluid pressure required to seal the through-hole.

199. The system of claim 194, wherein the first chamber is coupled to an injection device for injecting the fluid into the inlet chamber, and wherein the second chamber is coupled to a catheter configured to deliver the fluid to the isolated blood vessel region.

200. The system of claim 194, further comprising a bias member configured to bias the piston towards the open position.

201. The system of claim 200, wherein the valve is located in the second chamber.

202. The system of claim 201, wherein the valve is located in the second chamber.

203. The system of claim 194, wherein the piston also has a substantial frictional resistance to movement, and wherein the deflectable piston deflects away from the valve and towards the open position when the fluid pressure in the same chamber as the valve exceeds the bias on the deflectable piston and the substantial frictional resistance to movement.

204. An injection system for pressure regulated injection of a fluid into an isolated blood vessel region having a pressure regulator comprising:
a housing having a fluid output and slidably coupled with a plunger, the housing configured to house a fluid and dispense the fluid through the output upon depression of the plunger; and
the plunger comprising a plunger body, a bias member and a piston located between the housing and the bias member and configured to slide within the plunger body, wherein the bias member is configured to apply pressure to the piston in a first direction towards an extended state;
further wherein upon depression of the plunger, the fluid in the fluid housing applies pressure to the piston in a second direction opposite to the first direction such that the piston retracts from the extended state at a threshold point where the fluid pressure exceeds the bias member pressure.

205. The injection system of claim 204, further comprising a catheter coupled to the fluid output, the catheter configured to deliver the fluid to the isolated blood vessel region.

206. The injection system of claim 204, wherein the bias member is a cavity configured to contain a gas and the cavity comprises a pressure relief valve for releasing the gas at the threshold point.

207. The injection system of claim 204, wherein the bias member is configured to return the piston to the extended state once the fluid pressure in the housing is below the pressure applied by the bias member.

208. The injection system of claim 207, wherein the bias member is a spring.

209. The injection system of claim 204, further comprising an adjustment device adjustably coupled with the plunger body and the bias member and configured to adjust the pressure applicable by the bias member.

210. The injection system of claim 204, wherein the bias member is located in an inner cavity of the plunger body.

211. The injection system of claim 210, wherein the piston comprises an O-ring for sealing the inner cavity from the fluid housing.

212. The injection system of claim 210, wherein the bias member is a bellows and is further configured to seal the inner cavity from the fluid housing.

213. The injection system of claim 210, wherein the piston is coupled to the plunger body with a roller diaphragm further configured to seal the inner cavity from the fluid housing.

214. An injection system for pressure regulated injection of a fluid into an isolated blood vessel region having a pressure regulator comprising:
an injection device having a first fluid output and configured to output fluid from the first fluid output;
an output port having a second fluid output; and
an expandable bellows coupled with the first fluid output and the output port, the bellows configured to expand and accumulate the fluid output from the injection device when the fluid pressure at the first fluid output exceeds a threshold point defined by a resistance to expansion by the bellows and the fluid pressure exerted on the piston and bellows to expand.

215. The system of claim 214, wherein the bellows is configured to compress and output the accumulated fluid when the fluid pressure decreases to a level where the tendency of the bellows to compress exceeds the fluid pressure exerted on the output port and bellows to expand.

216. An injection system for pressure regulated injection of a fluid into an isolated blood vessel region having a pressure regulator comprising:
an injection device having a first fluid output and configured to output fluid from the first fluid output;
an output port having a second fluid output and a sleeve configured to slide over the first fluid output such that when the sleeve slides away from the first fluid output a cavity is created; and
a spring coupled with the first fluid output and the output port, the spring configured to extend and slide the sleeve away from the first fluid output and accumulate the fluid output from the injection device in the cavity when the fluid pressure at the first fluid output exceeds a threshold point defined by a resistance to extension by the spring and the fluid pressure exerted on the output port to extend.

217. The system of claim 216, wherein the spring is configured to compress and output the accumulated fluid when the fluid pressure decreases to a level where the tendency of the spring to compress exceeds the fluid pressure exerted on the output port to slide the sleeve away from the first fluid output.
